(19) United States
(12) Patent Application Publication
(10) Pub. No.: US 2017/0198119 A1
(43) Pub. Date: Jul. 13, 2017

(54) COPOLYMERS INCLUDING A TRIAZINE GROUP AND COMPOSITIONS INCLUDING THEM

(71) Applicant: 3M INNOVATIVE PROPERTIES COMPANY, St. Paul, MN (US)

(72) Inventors: David B. Olson, Hudson, WI (US); Patricia M. Savu, Maplewood, MN (US); Diane North, Inver Grove Heights, MN (US)

(21) Appl. No.: 15/320,562

(22) PCT Filed: Jun. 25, 2015
(86) PCT No.: PCT/US2015/037730

§ 371 Date: Dec. 20, 2016

Related U.S. Application Data
(60) Provisional application No. 62/017,021, filed on Jun. 25, 2014.
(60) Provisional application No. 62/017,666, filed on Jun. 26, 2014.

(51) Int. Cl. C08K 5/3492; C07D 251/24; C09K 15/30; C09J 127/16; C08F 214/22; C08J 5/18; C09J 11/06

(52) U.S. Cl. CPC C08K 5/3492; C08J 5/18; C07D 251/24; C09J 11/06; C09J 127/16; C08F 214/225; C09K 15/30; C08J 2327/16

(57) ABSTRACT

An ultraviolet light-absorbing oligomer that includes a first divalent unit represented by formula (I): and a second divalent unit represented by formula (II): Each R¹ is independently hydrogen or methyl; V is O or NH; X is bond or X is alkylene or alkyleneoxy group having from 1 to 10 carbon atoms and optionally interrupted by one or more —O— groups and optionally substituted by a hydroxyl group; and R2 is alkyl having from 1 to 22 carbon atoms. Compositions that include fluoropolymers and the oligomers are disclosed. The composition can be an extruded film. Compositions that include pressure sensitive adhesives and these oligomers are disclosed.

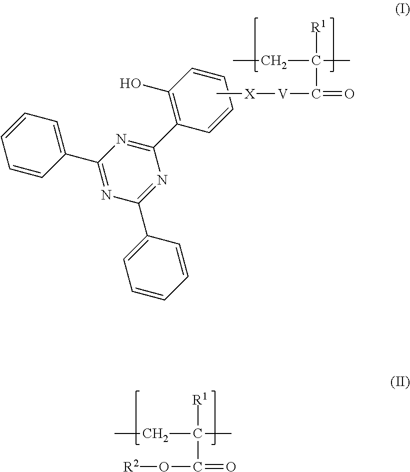

### CROSS-REFERENCE TO RELATED APPLICATION

This application claims priority to U.S. Provisional Application No. 62/017,021, filed Jun. 25, 2014, and 62/017,666, filed Jun. 26, 2014, the disclosures of which are incorporated by reference in their entirety herein.

### BACKGROUND

Fluoropolymers are known to have a variety of useful properties, including cleanability, weather resistance, and chemical resistance. Such beneficial properties render fluoropolymers useful, for example, for a variety of outdoor applications including signage, films or coatings for architectural coverings, and protective coverings for photovoltaic modules.

It may be desirable to incorporate ultraviolet absorbers (UVAs) into materials exposed to ultraviolet (UV) radiation, for example, to protect a topcoat or topsheet or an underlying substrate or adhesive from UV degradation. Some UVAs can be dispersed into some compositions, but sometimes they can be lost due to volatilization or migration to the surface. Covalent incorporation of UVAs into certain compositions has been proposed. See, e.g., U.S. Pat. Appl. Pub. No. 2011/0297228 (Li et al.).

It has been reported that common UVAs can be incompatible with fluoropolymers. See, e.g., U.S. Pat. No. 6,251,521 (Eian et al.). This incompatibility can lead to degradation of physical or optical properties (e.g., loss of clarity or increased fogginess) as well as increased or accelerated loss of the UVA by migration, bleeding, or blooming.

### SUMMARY

The present disclosure provides an oligomer having a first divalent unit with a pendent triazine group and compositions that include the oligomer. The composition may include a fluoropolymer. The oligomers are generally quite compatible with fluoropolymers such that the oligomers and fluoropolymers are readily blended together. Compositions including the fluoropolymers and oligomers provide protection from ultraviolet light and have good transparency to visible and infrared light. These properties are surprisingly well-maintained even after accelerated UV exposure and exposure to high temperature and humidity conditions.

In one aspect, the present disclosure provides a composition that includes a blend of a fluoropolymer and an ultraviolet light-absorbing oligomer. The ultraviolet light-absorbing oligomer includes a first divalent unit represented by formula:

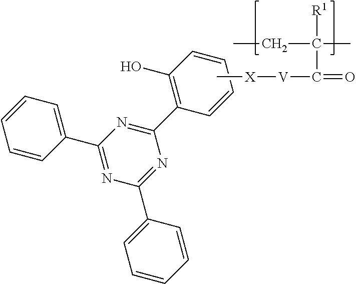

and a second divalent unit represented by formula:

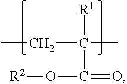

in which R¹ is hydrogen or methyl, V is O or NH; X is a bond, alkylene, or alkyleneoxy, wherein the alkylene and alkyleneoxy have from 1 to 10 carbon atoms and are optionally interrupted by one or more —O— groups and optionally substituted by a hydroxyl group, and R² is alkyl having from 1 to 4 carbon atoms.

In another aspect, the present disclosure provides an article that includes the composition. The article may be, for example, a photovoltaic device, vehicle wrap, graphic film, architectural film, or window film.

In another aspect, the present disclosure provides an ultraviolet light-absorbing oligomer including a first divalent unit represented by formula:

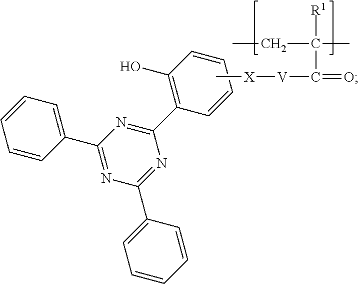

and

a second divalent unit represented by formula:

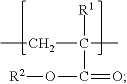

in which R¹ is hydrogen or methyl; V is O or NH; X is a bond, alkylene, or alkyleneoxy, wherein the alkylene and alkyleneoxy have from 1 to 10 carbon atoms and are optionally interrupted by one or more —O— groups and optionally substituted by a hydroxyl group; and R² is alkyl having from 1 to 22 carbon atoms.

In another aspect, the present disclosure provides a pressure sensitive adhesive including the ultraviolet light-absorbing oligomer.

In fluoropolymer compositions including an ultraviolet light-absorbing oligomer with a first divalent unit having a pendent ultraviolet absorbing group and a second divalent unit, the retention of the ultraviolet light-absorbing oligomers disclosed herein after exposure to ultraviolet light is generally much superior to the retention of conventional ultraviolet light absorbers after exposure to the same conditions. Unexpectedly, the retention of the ultraviolet light-absorbing oligomers after exposure to ultraviolet light generally is remarkably even better when the ultraviolet light-absorbing oligomer disclosed herein is used in comparison to structurally very similar oligomers in which the phenyl groups are substituted.

In this application:

Terms such as “a”, “an” and “the” are not intended to refer to only a singular entity, but include the general class of which a specific example may be used for illustration. The terms “a”, “an”, and “the” are used interchangeably with the term “at least one”.

The phrase “comprises at least one of” followed by a list refers to comprising any one of the items in the list and any combination of two or more items in the list. The phrase “at least one of” followed by a list refers to any one of the items in the list or any combination of two or more items in the list.

The term “ultraviolet absorbing group” or ultraviolet light-absorbing group refers to a covalently attached ultraviolet absorber (UVA). UVAs are known to those skilled in the art as being capable of dissipating absorbed light energy from UV rays as heat by reversible intramolecular proton transfer. UVAs are selected such that the oligomers in any of the embodiments of oligomers or second oligomers disclosed herein absorbs at least 70%, 80%, or 90% of incident light in a wavelength range from 180 nanometers (nm) to 400 nm.

“Alkyl group” and the prefix “alk-” are inclusive of both straight chain and branched chain groups and of cyclic groups. Unless otherwise specified, alkyl groups herein have up to 20 carbon atoms. Cyclic groups can be monocyclic or polycyclic and, in some embodiments, have from 3 to 10 ring carbon atoms.

The phrase “interrupted by at least one —O— group”, for example, with regard to an alkyl (which may or may not be fluorinated), alkylene, or arylalkylene refers to having part of the alkyl, alkylene, or arylalkylene on both sides of the —O— group. For example, —CH₂CH₂—O—CH₂—CH₂— is an alkylene group interrupted by an —O—.

The term “fluoroalkyl group” includes linear, branched, and/or cyclic alkyl groups in which all C—H bonds are replaced by C—F bonds as well as groups in which hydrogen or chlorine atoms are present instead of fluorine atoms. In some embodiments, up to one atom of either hydrogen or chlorine is present for every two carbon atoms.

The term “polymer” refers to a molecule having a structure which essentially includes the multiple repetition of units derived, actually or conceptually, from molecules of low relative molecular mass. The term “polymer” encompasses oligomers.

All numerical ranges are inclusive of their endpoints and nonintegral values between the endpoints unless otherwise stated (e.g., 1 to 5 includes 1, 1.5, 2, 2.75, 3, 3.80, 4, 5, etc.).

### DETAILED DESCRIPTION

Ultraviolet light-absorbing oligomers useful in the compositions according to the present disclosure are linear or branched. Typically, they are linear oligomers. They may be random copolymers or block copolymers. They are not covalently crosslinked. Accordingly, they may be dissolved in solvents and have measurable molecular weights as opposed to covalently crosslinked polymers, which cannot be dissolved in solvents and have molecular weights approaching infinity. In some embodiments, the oligomers may be considered thermoplastic. Thermoplastics are typically melt-processable such as by an extrusion process. Oligomers useful in the compositions according to the present disclosure have a number average molecular weight of up to 150,000 grams per mole. In some of these embodiments, the oligomer has a number average molecular weight of up to 120,000, 100,000, 90,000, 80,000, 70,000, 60,000, 50,000, 40,000, 30,000, 20,000, or less than 20,000 grams per mole (e.g., up to 19,500, 19,000, or 18,500 grams per mole). In some embodiments, the number average molecular weight of the oligomer may be at least 1000 grams per mole, greater than 5,000 grams per mole, or greater than 7,500 grams per mole. Useful ultraviolet light-absorbing oligomers typically have a distribution of molecular weights and compositions. Weight and number average molecular weights can be measured, for example, by gel permeation chromatography (i.e., size exclusion chromatography) using techniques known to one of skill in the art.

Ultraviolet light-absorbing oligomers useful in the compositions according to the present disclosure in any of their embodiments include a first divalent unit comprising a pendent ultraviolet absorbing triazine group. In some embodiments, the pendent ultraviolet absorbing group has enhanced spectral coverage in the long-wave UV region (e.g., 315 nm to 400 nm), enabling it to block the high wavelength UV light that can cause yellowing in polymers. The first divalent unit can be considered to be a repeating unit in the ultraviolet absorbing oligomer.

The ultraviolet light-absorbing oligomer according to the present disclosure and/or useful for practicing the present disclosure may include (e.g., at least 2, 3, 4, 5, 6, 7, 8, 9, 10, 15, or at least 20 up to 30, 35, 40, 45, 50, 100, 200, or up to 500 or more) independently selected first divalent units. The first divalent unit is represented by formula:

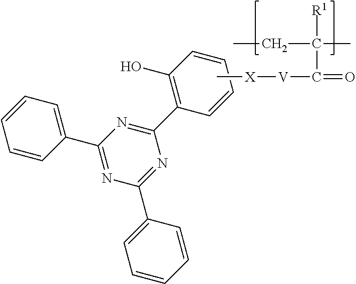

wherein R¹ is hydrogen or methyl, V is O or NH, X is a bond or X is alkylene or alkyleneoxy group having from 1 to 10 (in some embodiments, 2 to 6 or 2 to 4) carbon atoms and optionally interrupted by one or more —O— groups and optionally substituted by a hydroxyl group. In the alkyleneoxy group, the oxygen is attached to the substituted benzene ring. In some embodiments, each V is O, and X is ethylene, propylene, butylene, ethyleneoxy, propyleneoxy, or butyleneoxy, with the oxygen attached to the substituted benzene ring. In some embodiments, each V is O, and X is ethyleneoxy, propyleneoxy, or butyleneoxy, with the oxygen attached to the substituted benzene ring.

Ultraviolet light-absorbing oligomers according to the present disclosure and/or useful in the compositions according to the present disclosure comprise at least one (e.g., at least 2, 3, 4, 5, 6, 7, 8, 9, 10, 15, or at least 20 up to 30, 35, 40, 45, 50, 100, 200, 500, 1000, or up to 1500 or more) second divalent unit independently represented by formula:

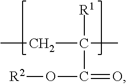

wherein each R¹ is independently hydrogen or methyl (in some embodiments, hydrogen, in some embodiments, methyl), and wherein each R² is independently alkyl having from 1 to 22 carbon atoms. In some embodiments, each R² is independently alkyl having from 1 to 20, 1 to 18, 1 to 16, 1 to 12, 1 to 8, or 1 to 4 carbon atoms. In some embodiments of the composition according to the present disclosure that includes a blend of a fluoropolymer and the ultraviolet light-absorbing oligomer, each R² in the second divalent units is independently alkyl having from 1 to 4 carbon atoms (in some embodiments, methyl, ethyl, n-propyl, isopropyl, n-butyl, iso-butyl, or tert-butyl). In some embodiments, each R² is independently methyl or ethyl. In some embodiments, each R² is methyl. In some embodiments, both R¹ and R² are methyl. In some embodiments of the composition according to the present disclosure that includes a blend of a pressure sensitive adhesive and the ultraviolet light-absorbing oligomer, each R² in the second divalent units is independently alkyl having from 4 to 20, 4 to 18, 4 to 16, or 4 to 12 carbon atoms. In some of these embodiments, R² has 8 carbon atoms (e.g., R² is ethylhexyl or isooctyl).

Ultraviolet light-absorbing oligomers according to the present disclosure and/or useful in the compositions according to the present disclosure can include other divalent units. In some embodiments, ultraviolet light-absorbing oligomers according to the present disclosure and/or useful in the compositions according to the present disclosure comprise at least one (e.g., at least 2, 3, 4, 5, 6, 7, 8, 9, 10, 15, or at least 20 up to 30, 35, 40, 45, 50, 100, 200, or up to 500 or more) third divalent unit independently represented by formula:

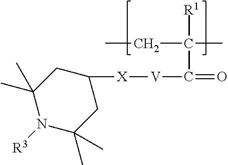

wherein each R¹ is independently hydrogen or methyl (in some embodiments, hydrogen, in some embodiments, methyl), V is O or NH, X is a bond or X is alkylene or alkyleneoxy group having from 1 to 10 (in some embodiments, 2 to 6 or 2 to 4) carbon atoms and optionally interrupted by one or more —O— groups and optionally substituted by a hydroxyl group, and R³ is hydrogen, alkyl, oxy, alkoxy (that is, —O— alkyl with the oxygen atom attached to the nitrogen atom), or alkanone (that is, —C(O)-alkyl with the carbonyl group attached to the nitrogen atom). In some embodiments, R³ is hydrogen or alkyl. In some embodiments, X is a bond. In some embodiments, X is an alkyleneoxy group. In the alkyleneoxy group, the oxygen is attached to the substituted piperidine ring. In some embodiments, each V is O and X is ethylene, propylene, butylene, ethyleneoxy, propyleneoxy, or butyleneoxy, with the oxygen attached to the substituted piperidine ring. It should be understood that when X is a bond, then the third divalent unit can be represented by formula:

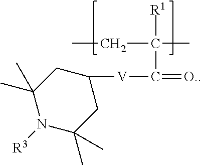

The tetramethylpiperidine group in the third divalent units can be useful as a hindered amine light stabilizer (HALS). In some embodiments, particularly in some of the Examples, below, the third divalent unit is referred to as the HALS group. HALS are typically compounds that can scavenge free-radicals, which can result from photodegradation.

In some embodiments, ultraviolet light-absorbing oligomers according to the present disclosure and/or useful in the compositions according to the present disclosure in any of the embodiments described above include (e.g., at least 1, 2, 3, 4, 5, 6, 7, 8, 9, 10, 15, or at least 20 up to 30, 35, 40, 45, 50, 100, 200, or up to 500 or more) fourth divalent units independently represented by formula:

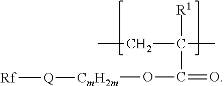

Incorporation of the fourth divalent units may be useful, for example, when the ultraviolet light-absorbing oligomer is incorporated into a blend including a fluoropolymer in a composition according to the present disclosure. For divalent units having this formula, each R′ is independently hydrogen or methyl (in some embodiments, hydrogen, in some embodiments, methyl). Q is a bond, —SO₂N(R)—, or —C(O)—N(R)— wherein R is alkyl having 1 to 4 carbon atoms (e.g., methyl, ethyl, n-propyl, isopropyl, n-butyl, or isobutyl) or hydrogen. In some embodiments, Q is a bond. In some embodiments, Q is —SO₂N(R)—. In some of these embodiments, R is methyl or ethyl. m is an integer from 1 to 11 (i.e., 1, 2, 3, 4, 5, 6, 7, 8, 9, 10, or 11). In some of these embodiments, m is 1; in other of these embodiments, m is 2. In some embodiments wherein Q is —SO₂N(R)—, m is an integer from 2 to 11, 2 to 6, or 2 to 4. In some embodiments wherein Q is a bond, m is an integer from 1 to 6, 1 to 4, or 1 to 2. In embodiments wherein Q is a bond, it should be understood that the fourth divalent units may also be represented by formula:

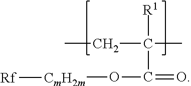

In some embodiments, oligomers disclosed herein, including any of the embodiments described above in connection to the first divalent units, comprise (e.g., at least 2, 3, 4, 5, 6, 7, 8, 9, 10, 15, or at least 20 up to 30, 35, 40, 45, 50, 100, 200, or up to 500 or more) fourth divalent units independently represented by formula:

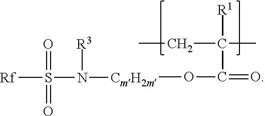

For divalent units of this formula, m′ is an integer from 2 to 11 (i.e., 2, 3, 4, 5, 6, 7, 8, 9, 10, or 11). In some embodiments, m′ is an integer from 2 to 6 or 2 to 4. R³ is alkyl having 1 to 4 carbon atoms (e.g., methyl, ethyl, n-propyl, isopropyl, n-butyl, or isobutyl) or hydrogen. In some embodiments, R³ is methyl or ethyl. R′ is independently hydrogen or methyl (in some embodiments, hydrogen, in some embodiments, methyl).

For any of the embodiments of the fourth divalent units, each Rf independently represents a fluorinated alkyl group having from 1 to 6 (in some embodiments, 2 to 6 or 2 to 4) carbon atoms (e.g., trifluoromethyl, perfluoroethyl, 1,1,2,2-tetrafluoroethyl, 2-chlorotetrafluoroethyl, perfluoro-n-propyl, perfluoroisopropyl, perfluoro-n-butyl, 1,1,2,3,3,3-hexafluoropropyl, perfluoroisobutyl, perfluoro-sec-butyl, or perfluoro-tert-butyl, perfluoro-n-pentyl, pefluoroisopentyl, or perfluorohexyl). In some embodiments, Rf is perfluorobutyl (e.g., perfluoro-n-butyl, perfluoroisobutyl, or perfluoro-sec-butyl). In some embodiments, Rf is perfluoropropyl (e.g., perfluoro-n-propyl or perfluoroisopropyl). The oligomer may include a mixture of fluorinated monomers having different Rf fluoroalkyl groups (e.g., with an average of up to 6 or 4 carbon atoms).

In some embodiments, in oligomers disclosed herein, including any of the embodiments described above in connection to the first, second, and third divalent units, Rf is a polyfluoroether group. The term “polyfluoroether” refers to a compound or group having at least 3 (in some embodiments, at least 4, 5, 6, 7, 8, 9, 10, 11, 12, 13, 14, 15, 16, 17, 18, 19, or even 20) carbon atoms and at least 1 (in some embodiments, at least 2, 3, 4, 5, 6, 7, or even 8) ether linkages, wherein hydrogen atoms on the carbon atoms are replaced with fluorine atoms. In some embodiments, Rf has up to 100, 110, 120, 130, 140, 150, or even 160 carbon atoms and up to 25, 30, 35, 40, 45, 50, 55, or even 60 ether linkages.

In some embodiments, including embodiments wherein Rf is a polyfluoroether group, oligomers disclosed herein comprise (e.g., at least 2, 3, 4, 5, 6, 7, 8, 9, 10, 15, or at least 20 up to 30, 35, 40, 45, 50, 100, 200, or up to 500 or more) fourth divalent units independently represented by formula:

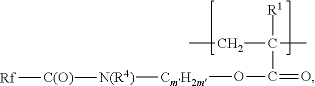

For divalent units of this formula, m′ is an integer from 2 to 11 (i.e., 2, 3, 4, 5, 6, 7, 8, 9, 10, or 11). In some embodiments, m′ is an integer from 2 to 6 or 2 to 4. R⁴ is alkyl having 1 to 4 carbon atoms (e.g., methyl, ethyl, n-propyl, isopropyl, n-butyl, or isobutyl) or hydrogen. In some embodiments, R⁴ is methyl or ethyl. In some embodiments, R⁴ is hydrogen. R′ is independently hydrogen or methyl (in some embodiments, hydrogen, in some embodiments, methyl).

The polyfluoroether group Rf can be linear, branched, cyclic, or combinations thereof and can be saturated or unsaturated. Polyfluoroether groups include those in which hydrogen or chlorine atoms are present instead of fluorine atoms with typically up to one atom of either hydrogen or chlorine is present for every two carbon atoms. The oligomer may include a mixture of fluorinated monomers having different Rf polyfluoroether groups. In some embodiments, the polyfluoroether group is a perfluoropolyether group (i.e., all of the hydrogen atoms on the carbon atoms are replaced with fluorine atoms). Exemplary perfluoropolyethers include perfluorinated repeating units represented by at least one of —(C_{d}F_{2d})—, —(C_{d}F_{2d}O)—, —(CF(L′))-, —(CF(L′)O)—, —(CF(L′)C_{d}F_{2d}O)—, —(C_{d}F_{2d}CF(L′)O)—, or —(CF₂CF(L′)O)—. In these repeating units, d is typically an integer from 1 to 10. In some embodiments, d is an integer from 1 to 8, 1 to 6, 1 to 4, or 1 to 3. The L′ group can be a perfluoroalkyl group optionally interrupted by at least one ether linkage or a perfluoroalkoxy group, each of which may be linear, branched, cyclic, or a combination thereof. The L′ group typically has up to 12 (in some embodiments, up to 10, 8, 6, 4, 3, 2, or 1) carbon atoms. In some embodiments, the L′ group can have up to 4 (in some embodiments, up to 3, 2, or 1) oxygen atoms; in some embodiments L′ has no oxygen atoms. In these perfluoropolyether structures, different repeating units can be combined in a block or random arrangement to form the Rf group.

In some embodiments, Rf is represented by formula R_{f}^{a}—O—(R_{f}^{b}—O—)_{z′}(R_{f}^{c})—, wherein R_{f}^{a} is a perfluoroalkyl having 1 to 10 (in some embodiments, 1 to 6, 1 to 4, 2 to 4, or 3) carbon atoms; each R_{f}^{b} is independently a perfluoroalkylene having 1 to 4 (i.e., 1, 2, 3, or 4) carbon atoms; R_{f}^{c} is a perfluoroalkylene having 1 to 6 (in some embodiments, 1 to 4 or 2 to 4) carbon atoms; and z′ is in a range from 2 to 50 (in some embodiments, 2 to 25, 2 to 20, 3 to 20, 3 to 15, 5 to 15, 6 to 10, or 6 to 8). Representative RP groups include CF₃—, CF₃CF₂—, CF₃CF₂CF₂—, CF₃CF(CF₃)—, CF₃CF(CF₃)CF₂—, CF₃CF₂CF₂CF₂—, CF₃CF₂CF(CF₃)—, CF₃CF₂CF(CF₃)CF₂—, and CF₃CF(CF₃)CF₂CF₂—. In some embodiments, R_{f}^{a} is CF₃CF₂CF₂—. Representative R_{f}^{b} groups include —CF₂—, —CF(CF₃)—, —CF₂CF₂—, —CF(CF₃)CF₂—, —CF₂CF₂CF₂—, —CF(CF₃)CF₂CF₂—, —CF₂CF₂CF₂CF₂—, and —CF₂C(CF₃)₂—. Representative R_{f}^{c} groups include —CF₂—, —CF(CF₃)—, —CF₂CF₂—, —CF₂CF₂CF₂—, and —CF(CF₃)CF₂—. In some embodiments, R_{f}^{c} is —CF(CF₃)—.

In some embodiments, (R_{f}^{b}—O—)_{z′} is represented by —[CF₂O]ᵢ[CF₂CF₂O]ⱼ—, —[CF₂O]ᵢ[CF(CF₃)CF₂O]ⱼ—, —[CF₂O]ᵢ[CF₂CF₂CF₂O]ⱼ—, —[CF₂CF₂O]ᵢ[CF₂O]ⱼ—, —[CF₂CF₂O]ᵢ[CF(CF₃)CF₂O]ⱼ—, —[CF₂CF₂O]ᵢ[CF₂CF₂CF₂O]ⱼ—, —[CF₂CF₂CF₂O]ᵢ[CF₂CF(CF₃)O]ⱼ—, and [CF₂CF₂CF₂O]ᵢ[CF(CF₃)CF₂O]ⱼ—, wherein i+j is an integer of at least 3 (in some embodiments, at least 4, 5, or 6).

In some embodiments, Rf is selected from the group consisting of C₃F₇O(CF(CF₃)CF₂O)ₖCF(CF₃)—, C₃F₇O(CF₂CF₂CF₂O)ₖCF₂CF₂—, or CF₃O(C₂F₄O)_{g}CF₂—, wherein k has an average value in a range from 3 to 50 (in some embodiments, 3 to 25, 3 to 15, 3 to 10, 4 to 10, or 4 to 7), and wherein g has an average value in a range from 6 to 50 (in some embodiments, 6 to 25, 6 to 15, 6 to 10, 7 to 10, or 8 to 10). In some of these embodiments, Rf is C₃F₇O(CF(CF₃)CF₂O)ₖCF(CF₃)—, wherein k has an average value in a range from 4 to 7. In some embodiments, Rf is selected from the group consisting of CF₃O(CF₂O)_{x′}(C₂F₄O)_{y′}CF₂— and F(CF₂)₃—O—(C₄F₈O)_{z′}(CF₂)₃—, wherein x′, y′, and z′ each independently has an average value in a range from 3 to 50 (in some embodiments, 3 to 25, 3 to 15, 3 to 10, or even 4 to 10).

In some embodiments, Rf is a polyfluoropolyether group that has a weight average molecular weight of at least 750 (in some embodiments at least 850 or even 1000) grams per mole. In some embodiments, Rf has a weight average molecular weight of up to 6000 (in some embodiments, 5000 or even 4000) grams per mole. In some embodiments, Rf has a weight average molecular weight in a range from 750 grams per mole to 5000 grams per mole. Weight average molecular weights can be measured, for example, by gel permeation chromatography (i.e., size exclusion chromatography) using techniques known in the art.

In some embodiments, ultraviolet light-absorbing oligomers according to the present disclosure and/or useful in the compositions according to the present disclosure comprise at least one (e.g., at least 2, 3, 4, 5, 6, 7, 8, 9, 10, 15, or at least 20 up to 30, 35, 40, 45, 50, 100, 200, or up to 500 or more) fifth divalent unit independently represented by formula:

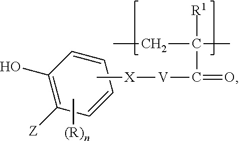

wherein R¹ is hydrogen or methyl, V is O or NH, X is a bond or X is alkylene or alkyleneoxy group having from 1 to 10 (in some embodiments, 2 to 6 or 2 to 4) carbon atoms and optionally interrupted by one or more —O— groups and optionally substituted by a hydroxyl group, R is alkyl (e.g., having from one to four carbon atoms), n is 0 or 1, and Z is a benzoyl group or a 2H-benzotriazol-2-yl group, wherein the benzoyl group or 2H-benzotriazol2-yl group is optionally substituted by one or more alkyl, aryl, alkoxy, hydroxyl, or halogen substituents, or a combination of these substituents. In some embodiments, the alkyl and/or alkoxy substituent independently has 1 to 4 or 1 to 2 carbon atoms. In some embodiments, each halogen substituent is independently a chloro, bromo, or iodo group. In some embodiments, each halogen substituent is a chloro group. The term “aryl” as used herein includes carbocyclic aromatic rings or ring systems, for example, having 1, 2, or 3 rings and optionally containing at least one heteroatom (e.g., 0, S, or N) in the ring. Examples of aryl groups include phenyl, naphthyl, biphenyl, fluorenyl as well as furyl, thienyl, pyridyl, quinolinyl, isoquinolinyl, indolyl, isoindolyl, triazolyl, pyrrolyl, tetrazolyl, imidazolyl, pyrazolyl, oxazolyl, and thiazolyl. In the alkyleneoxy group, the oxygen is attached to the substituted benzene ring. In some embodiments, each V is O, and X is ethylene, propylene, butylene, ethyleneoxy, propyleneoxy, or butyleneoxy, with the oxygen attached to the substituted benzene ring. In some embodiments, each V is O, and X is ethyleneoxy, propyleneoxy, or butyleneoxy, with the oxygen attached to the substituted benzene ring. In some embodiments, n is O. In some embodiments, R is methyl, ethyl, n-propyl, isopropyl, n-butyl, isobutyl, s-butyl, or t-butyl, and n is 1. In some embodiments, Z is an unsubstituted benzoyl group. In some embodiments, Z is 2H-benzotriazol-2-yl or 5-chloro-2H-benzotriazol-2-yl. In some embodiments, Z can also be a substituted 4,6-bisphenyl-[1,3,5]triazin-2-yl group. In some of these embodiments, Z is 4,6-bis(2,4-dimethylphenyl)[1,3,5]triazin-2-yl; 4,6-bis(2,4-diethylphenyl)[1,3,5]triazin-2-yl; 4,6-bis(2, 4-dimethoxyphenyl)[1,3,5]triazin-2-yl; or 4,6-bis(2,4-diethoxyphenyl)[1,3,5]triazin-2-yl.

In some embodiments, ultraviolet light-absorbing oligomers according to the present disclosure and/or useful in the compositions according to the present disclosure comprise at least one (e.g., at least 2, 3, 4, 5, 6, 7, 8, 9, 10, 15, or at least 20 up to 30, 35, 40, 45, 50, 100, 200, 500, or up to 1000) sixth divalent unit comprising a pendent carboxylic acid, hydroxyl, or aminocarbonyl group. The aminocarbonyl group can be aminocarbonyl (—C(O)—NH₂), alkylaminocarbonyl, dialkylaminocarbonyl, wherein the alkyl in the alkylaminocarbonyl or dialkylaminocarbonyl is optionally substituted by hydroxyl. It will be understood by a person skilled in the art that an aminocarbonyl group is also known as an amido group. When more than one sixth divalent unit is present, the sixth divalent units may be independently selected.

When any of the first, second, third, fourth, fifth, and sixth divalent units are present, each R′ is independently selected.

Oligomers according to the present disclosure can be prepared, for example, by polymerizing a mixture of components typically in the presence of an initiator. By the term “polymerizing” it is meant forming a polymer or oligomer that includes at least one identifiable structural element due to each of the components. Typically, preparing the ultraviolet light-absorbing oligomer includes combining components comprising at least a first monomer having 4,6-bisphenyl-[1,3,5]triazin-2-yl group, a second monomer, and optionally at least one of a third, fourth, fifth, or sixth monomer described below. Suitable first monomers include 2,4-diphenyl-6-[2-hydroxy-4-(2-acryloyloxyethoxy)]-1,3,5-triazine and 2,4-diphenyl-6-[2-hydroxy-4-(2-methacryloyloxyethoxy)]-1,3,5-triazine. Suitable first monomers can be prepared by treating a 2,4-diphenyl-6-(2,4-dihydroxy)-1,3,5-triazine with (meth)acrylic acid or an equivalent thereof using conventional esterification methods. The term (meth)acrylic refers to both acrylic and methacrylic. In some embodiments, the phenol group not ortho to the triazine group may be treated with ethylene carbonate or ethylene oxide to form a hydroxyethyl group that can then be treated with (meth)acrylic acid or an equivalent thereof using conventional esterification methods.

The components that are useful for preparing the oligomers disclosed herein include a second monomer. In some of these embodiments, the oligomer is prepared by including at least one compound represented by formula R²—O—C(O)—C(R¹)═CH₂ as the second monomer in the components to be polymerized. R¹ and R² are as defined above in any of their embodiments. Suitable second monomers of this formula include methyl methacrylate, ethyl acrylate, propyl acrylate, butyl acrylate, isoamyl acrylate, ethylhexyl acrylate, isooctyl acrylate, nonyl acrylate, dodecyl acrylate, hexadecyl methacrylate, octadecyl methacrylate, stearyl acrylate, behenyl methacrylate, acrylates of the foregoing methacrylates and methacrylates of the foregoing acrylates. Many of these second monomers are available, for example, from several chemical suppliers (e.g., Sigma-Aldrich Company, Milwaukee, Wis.; VWR International, West Chester, Pa.; Monomer-Polymer & Dajac Labs, Festerville, Pa.; Avocado Organics, Ward Hill, Mass.; and Ciba Specialty Chemicals, Basel, Switzerland) or may be synthesized by conventional methods. Some of these second monomers are available as single isomers (e.g., straight-chain isomer) of single compounds. Other are available, for example, as mixtures of isomers (e.g., straight-chain and branched isomers), mixtures of compounds (e.g., hexadecyl acrylate and octadecylacrylate), and combinations thereof.

The components that are useful for preparing the ultraviolet light-absorbing oligomer according to the present disclosure and/or useful in the compositions according to the present disclosure can include a third monomer that includes a 2,2,6,6-tetramethylpiperidinyl group in which the nitrogen atom is substituted by hydrogen, alkyl, oxy, alkoxy, or alkanone. Examples of suitable third monomers include 2,2,6,6,-tetramethyl-4-piperidyl methacrylate, 1,2,2,6,6-pentamethyl-4-piperidyl methacrylate, 4-methacryloylamino-2,2,6,6-tetramethylpiperidine, 4-methacryloylamino-1,2,2,6,6-pentamethylpiperidine, 2,2,6,6,-tetramethyl-1-oxy-4-piperidyl methacrylate, 4-methacryloylamino-2,2,6,6-tetramethyl-1-oxypiperidine, 2,2,6,6,-tetramethyl-4-piperidyl acrylate, 1,2,2,6,6-pentamethyl-4-piperidyl acrylate, 4-acryloylamino-2,2,6,6-tetramethylpiperidine, 4-acryloylamino-1,2,2,6,6-pentamethylpiperidine, 2,2,6,6,-tetramethyl-1-oxy-4-piperidyl acrylate, and 4-acryloylamino-2,2,6,6-tetramethyl-1-oxypiperidine. Many of these first monomers can be obtained commercially from a variety of chemical suppliers. Others can be prepared by treating a 2,2,6,6-tetramethylpiperidine having an available hydroxyl group with (meth)acrylic acid or an equivalent thereof using conventional esterification methods. The term (meth)acrylic refers to both acrylic and methacrylic. For example, the hydroxyl group may be treated with (meth)acrylic acid or an equivalent thereof using conventional esterification methods.

The components that are useful for preparing the ultraviolet light-absorbing oligomer according to the present disclosure and/or useful in the compositions according to the present disclosure can include a fourth monomer, typically a fluorinated free-radically polymerizable monomer independently represented by formula Rf-Q-(CₘH₂ₘ)—O—C(O)—C(R¹)═CH₂, Rf—SO₂—N(R³)—(C_{m′}H_{2m′})—O—C(O)—C(R¹)═CH₂, or Rf—CO—N(R⁴)—(C_{m′}H_{2m′})—O—C(O)—C(R¹)═CH₂, wherein Rf, R³, R⁴, R¹, m, and m′ are as defined above.

Some compounds of Formula Rf-Q-(CₘH₂ₘ)—O—C(O)—C(R¹)═CH₂, are available, for example, from commercial sources (e.g., 3,3,4,4,5,5,6,6,6-nonafluorohexyl acrylate from Daikin Chemical Sales, Osaka, Japan; 3,3,4,4,5,5,6,6,6-nonafluorohexyl 2-methylacrylate from Indofine Chemical Co., Hillsborough, N.J.; 1H,1H,2H,2H-perfluorooctylacrylate from ABCR, Karlsruhe, Germany; and 2,2,3,3,4,4,5,5-octafluoropentyl acrylate and methacrylate and 3,3,4,4,5,6,6,6-octafluoro-5-(trifluoromethyl)hexyl methacrylate from Sigma-Aldrich, St. Louis, Mo.). Others can be made by known methods (see, e.g., EP1311637 B1, published Apr. 5, 2006, for the preparation of 2,2,3,3,4,4,4-heptafluorobutyl 2-methylacrylate). Compounds wherein Q is —SO₂N(R)— can be made according to methods described in, e.g., U.S. Pat. No. 2,803,615 (Albrecht et al.) and U.S. Pat. No. 6,664,354 (Savu et al.), the disclosures of which, relating to free-radically polymerizable monomers and methods of their preparation, are incorporated herein by reference. A perfluoropolyether monomer of formula Rf—(CO)NHCH₂CH₂O(CO)C(R¹)═CH₂ can be prepared by first reacting Rf—C(O)—OCH₃, for example, with ethanolamine to prepare alcohol-terminated Rf—(CO)NHCH₂CH₂OH, which can then be reacted with (meth)acrylic acid, (meth)acrylic anhydride, or (meth)acryloyl chloride to prepare the compound of Formula Rf—(CO)NHCH₂CH₂O(CO)C(R¹)═CH₂, wherein R′ is methyl or hydrogen, respectively. Other amino alcohols (e.g., amino alcohols of formula NRHXOH) can be used in this reaction sequence. In further examples, an ester of formula Rf—C(O)—OCH₃ or a carboxylic acid of formula Rf—C(O)—OH can be reduced using conventional methods (e.g., hydride, for example sodium borohydride, reduction) to an alcohol of formula Rf—CH₂OH. The alcohol of formula Rf—CH₂OH can then be reacted with methacryloyl chloride, for example, to provide a perfluoropolyether monomer of formula Rf—CH₂O(CO)C(R¹)═CH₂. Examples of suitable reactions and reagents are further disclosed, for example, in the European patent EP 870 778 A1, published Oct. 14, 1998, and U.S. Pat. No. 3,553,179 (Bartlett et al.).

Suitable fifth monomers for some embodiments of the compositions disclosed herein are those that include benzophenone, benzotriazole, cinnamate, cyanoacrylate, dicyano ethylene, salicylate, oxanilide, or para-aminobenzoate groups. In some embodiments, the fifth monomer includes a benzophenone or a benzotriazole group. Examples of suitable first monomers include 2-(cyano-β,β-biphenylacryloyloxy)ethyl-1-methacrylate, 2-(α-cyano-β,β-biphenylacryloyloxy)ethyl-2-methacrylamide, N-(4-methacryloylphenol)-N′-(2-ethylphenyl)oxamide, vinyl 4-ethyl-α-cyano-β-phenylcinnamate, 2-hydroxy-4-(2-hydroxy-3-methacryloyloxypropoxy)benzophenone, 2-hydroxy-4-methacryloyloxybenzophenone, 2-hydroxy-4-(2-acryloyloxyethoxy)benzophenone, 2-hydroxy-4-(4-acryloyloxybutoxy)benzophenone, 2,2′-dihydroxy-4-(2-acryloyloxyethoxy)benzophenone, 2-hydroxy-4-(2-acryloyloxyethoxy)-4′-(2-hydroxyethoxy)benzophenone, 4-(allyloxy)-2-hydroxybenzophenone, 2-(2′-hydroxy-3′-methacrylamidomethyl-5′-octylphenyl)benzotriazole, 2-(2-hydroxy-5-vinylphenyl)-2-benzotriazole, 2-(2H-benzotriazol-2-yl)-4-methyl-6-(2-propenyl)phenol, 2-(2′-hydroxy-5′-methacryloyloxyethylphenyl)-2H-benzotriazole, 2-(2′-hydroxy-5′-methacryloyloxyethylphenyl)-5-chloro-2H-benzotriazole, 2-(2′-hydroxy-5′-methacryloyloxypropylphenyl)-2H-benzotriazole, 2-(2′-hydroxy-5′-methacryloyloxypropylphenyl)-5-chloro-2H-benzotriazole, 2-(2′-hydroxy-3′-tert-butyl-5′-methacryloyloxyethylphenyl)-2H-benzotriazole, 2-(2′-hydroxy-3′-tertbutyl-5′-methacryloyloxyethylphenyl)-5-chloro-2H-benzotriazole, methacrylates of the foregoing acrylates and acrylates of the foregoing methacrylates. In some embodiments, suitable fifth monomers can also include substituted 2,4-diphenyl-1,3,5-triazine groups. Suitable fifth monomers of this type include 2,4-bis(2-methylphenyl)-6-[2-hydroxy-4-(2-acryloyloxyethoxy)]-1,3,5-triazine, 2,4-bis(2-methoxyphenyl)-6-[2-hydroxy-4-(2-acryloyloxyethoxy)]-1,3,5-triazine, 2,4-bis(2-ethylphenyl)-6-[2-hydroxy-4-(2-acryloyloxyethoxy)]-1,3,5-triazine, 2,4-bis(2-ethoxyphenyl)-6-[2-hydroxy-4-(2-acryloyloxyethoxy)]-1,3,5-triazine, 2,4-bis(2-methylphenyl)-6-[2-hydroxy-4-(2-methacryloyloxyethoxy)]-1,3,5-triazine, 2,4-bis(2-methoxyphenyl)-6-[2-hydroxy-4-(2-methacryloyloxyethoxy)]-1,3,5-triazine, 2,4-bis(2-ethylphenyl)-6-[2-hydroxy-4-(2-methacryloyloxyethoxy)]-1,3,5-triazine, 2,4-bis(2-ethoxyphenyl)-6-[2-hydroxy-4-(2-methacryloyloxyethoxy)]-1,3,5-triazine, 2,4-bis(2,4-dimethoxyphenyl)-6-[2-hydroxy-4-(2-acryloyloxyethoxy)]-1,3,5-triazine, 2,4-bis(2,4-dimethylphenyl)-6-[2-hydroxy-4-(2-acryloyloxyethoxy)]-1,3,5-triazine, 2,4-bis(2,4-diethoxyphenyl)-6-[2-hydroxy-4-(2-acryloyloxyethoxy)]-1,3,5-triazine, and 2,4-bis (2,4-diethylphenyl)-6-[2-hydroxy-4-(2-acryloyloxyethoxy)]-1,3,5-triazine. Combinations of these fifth monomers may be used to prepare the ultraviolet light-absorbing oligomer.

Many of these fifth monomers can be obtained commercially from a variety of chemical suppliers. Others can be prepared by treating a UVA having an available hydroxyl group (e.g., other than a phenolic hydroxyl group ortho to a triazine, benzoyl, or benzotriazole group) with (meth)acrylic acid or an equivalent thereof using conventional esterification methods. The term (meth)acrylic refers to both acrylic and methacrylic. In the case of a UVA having an available phenol group (e.g., other than a phenolic hydroxyl group ortho to a triazine, benzoyl, or benzotriazole group), the phenol group may be treated with ethylene carbonate or ethylene oxide to form a hydroxyethyl group that can then be treated with (meth)acrylic acid or an equivalent thereof using conventional esterification methods.

Suitable sixth monomers in some embodiments of the oligomers according to the present disclosure include an acrylic acid (e.g., acrylic acid, methacrylic acid, itaconic acid, maleic acid, and fumaric acid), a (meth)acrylamide (e.g., acrylamide, methacrylamide, N-ethyl acrylamide, N-hydroxyethyl acrylamide, N-octyl acrylamide, N-t-butyl acrylamide, N,N-dimethyl acrylamide, N,N-diethyl acrylamide, N-ethyl-N-dihydroxyethyl acrylamide, and methacrylamides of the foregoing acrylamides), a hydroxyalkyl (meth)acrylate (e.g., 2-hydroxyethyl acrylate or methacrylate, 3-hydroxypropyl acrylate or methacrylate, 4-hydroxybutyl acrylate or methacrylate, 8-hydroxyoctyl acrylate or methacrylate, or 9-hydroxynonyl acrylate or methacrylate). N-vinyl pyrrolidone and N-vinyl caprolactam may also be useful in the preparation of the ultraviolet light-absorbing oligomers disclosed herein.

In some embodiments, the ultraviolet light-absorbing oligomer according to the present disclosure and/or useful in the compositions according to the present disclosure is represented by formula:

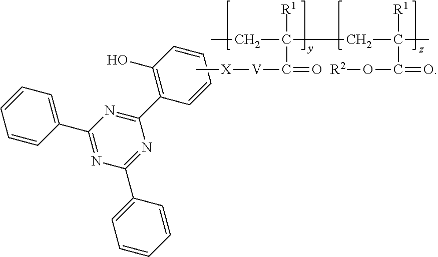

In this formula, X, V, R¹, and R² are as defined above in any of their embodiments and y and z are any of the ranges described above. It should be understood that the representation of the order of the divalent units in this formula is for convenience only and not meant to specify that the oligomers are block copolymers. Random copolymers having first and second divalent units are also included in the representation. The representation can also include any of the third, fourth, fifth, or sixth divalent units described above in any order.

The polymerization reaction for making the oligomers useful in the compositions according to the present disclosure can be carried out in the presence of an added free-radical initiator. Free radical initiators such as those widely known and used in the art may be used to initiate polymerization of the components. Examples of suitable free-radical initiators include azo compounds (e.g., 2,2′-azobisisobutyronitrile (AIBN), 2,2′-azobis(2-methylbutyronitrile), or azo-2-cyanovaleric acid), hydroperoxides (e.g., cumene, tert-butyl or tert-amyl hydroperoxide), dialkyl peroxides (e.g., di-tert-butyl or dicumylperoxide), peroxyesters (e.g., tert-butyl perbenzoate or di-tert-butyl peroxyphthalate), and diacylperoxides (e.g., benzoyl peroxide or lauryl peroxide).

The free-radical initiator may also be a photoinitiator. Examples of useful photoinitiators include benzoin ethers (e.g., benzoin methyl ether or benzoin butyl ether); acetophenone derivatives (e.g., 2,2-dimethoxy-2-phenylacetophenone or 2,2-diethoxyacetophenone); 1-hydroxycyclohexyl phenyl ketone; and acylphosphine oxide derivatives and acylphosphonate derivatives (e.g., bis(2,4,6-trimethylbenzoyl)phenylphosphine oxide, diphenyl-2,4,6-trimethylbenzoylphosphine oxide, isopropoxyphenyl-2,4,6-trimethylbenzoylphosphine oxide, or dimethyl pivaloylphosphonate). Many photoinitiators are available, for examples, from BASF, Florham Park, N.J., under the trade designation “IRGACURE”. The photoinitiator may be selected so that the wavelength of light required to initiate polymerization is not absorbed by the ultraviolet absorbing group.

In some embodiments, the polymerization reaction is carried out in solvent. The components may be present in the reaction medium at any suitable concentration, (e.g., from about 5 percent to about 80 percent by weight based on the total weight of the reaction mixture). Illustrative examples of suitable solvents include aliphatic and alicyclic hydrocarbons (e.g., hexane, heptane, cyclohexane), aromatic solvents (e.g., benzene, toluene, xylene), ethers (e.g., diethyl ether, glyme, diglyme, and diisopropyl ether), esters (e.g., ethyl acetate and butyl acetate), alcohols (e.g., ethanol and isopropyl alcohol), ketones (e.g., acetone, methyl ethyl ketone and methyl isobutyl ketone), halogenated solvents (e.g., methylchloroform, 1,1,2-trichloro-1,2,2-trifluoroethane, trichloroethylene, trifluorotoluene, and hydrofluoroethers available, for example, from 3M Company, St. Paul, Minn. under the trade designations “HFE-7100” and “HFE-7200”), and mixtures thereof.

Polymerization can be carried out at any temperature suitable for conducting an organic free-radical reaction. Temperature and solvent for a particular use can be selected by those skilled in the art based on considerations such as the solubility of reagents, temperature required for the use of a particular initiator, and desired molecular weight. While it is not practical to enumerate a particular temperature suitable for all initiators and all solvents, generally suitable temperatures are in a range from about 30° C. to about 200° C. (in some embodiments, from about 40° C. to about 100° C., or from about 50° C. to about 80° C.).

Free-radical polymerizations may be carried out in the presence of chain transfer agents. Typical chain transfer agents that may be used in the preparation compositions according to the present invention include hydroxyl-substituted mercaptans (e.g., 2-mercaptoethanol, 3-mercapto-2-butanol, 3-mercapto-2-propanol, 3-mercapto-1-propanol, and 3-mercapto-1,2-propanediol (i.e., thioglycerol)); poly(ethylene glycol)-substituted mercaptans; carboxy-substituted mercaptans (e.g., mercaptopropionic acid or mercaptoacetic acid): amino-substituted mercaptans (e.g., 2-mercaptoethylamine); difunctional mercaptans (e.g., di(2-mercaptoethyl)sulfide); and aliphatic mercaptans (e.g., octylmercaptan, dodecylmercaptan, and octadecylmercaptan).

Adjusting, for example, the concentration and activity of the initiator, the concentration of each of the reactive monomers, the temperature, the concentration of the chain transfer agent, and the solvent using techniques known in the art can control the molecular weight of the oligomer.

The weight ratio of the first divalent units, second divalent units, and any of the third, fourth, fifth, or sixth divalent units in the oligomers disclosed herein in any of their embodiments may vary. For example, the first divalent units may be present in the ultraviolet light-absorbing oligomer in a range from 5 to 50 (in some embodiments, 10 to 40 or 10 to 30) percent, based on the total weight of the oligomer. The second divalent units may be present in a range from 5 to 95 percent, based on the total weight of the oligomer. In some embodiments, the second divalent unit is present in the oligomer in an amount of up to 90, 80, 75, or 70 percent by weight, based on the total weight of the oligomer.

When the third divalent unit is present in the ultraviolet light-absorbing oligomer, the third divalent unit may be present in a range from 1 to 25, 2 to 20, or 5 to 15 percent by weight, based on the total weight of the oligomer.

When the fourth divalent unit is present in the ultraviolet light-absorbing oligomer, it may be present in a range from 5 to 90, 10 to 90, 20 to 90, or 10 to 50 percent by weight, based on the total weight of the oligomer. When the fourth divalent unit is present in the ultraviolet light-absorbing oligomer in an amount of at least 50, 60, 75, or 80 percent, it may be useful to use the oligomer in combination with another oligomer having a lower weight percentage of fourth divalent units.

When the fifth divalent unit is present in the ultraviolet light-absorbing oligomer, the first and fifth divalent units may be present in the ultraviolet light-absorbing oligomer in a range from 5 to 50 (in some embodiments, 10 to 40 or 10 to 30) percent, based on the total weight of the oligomer. The fifth divalent unit itself may be present in a range from 1 to 25, 2 to 20, or 1 to 15 percent by weight, based on the total weight of the oligomer.

When the sixth divalent unit is present in the ultraviolet light-absorbing oligomer, the sixth divalent unit may be present in a range from 1 to 15, 1 to 10, or 1 to 5 percent by weight, based on the total weight of the oligomer.

It can be useful to have a second, different oligomer in addition to the ultraviolet light-absorbing oligomers in compositions according to the present disclosure, for example, a fluoropolymer composition or a pressure sensitive adhesive composition described below. The second, different oligomer includes the second divalent unit and at least one of a third divalent unit comprising a pendent 2,2,6,6-tetramethylpiperidinyl group, wherein the nitrogen of the pendent 2,2,6,6-tetramethylpiperidinyl group is substituted by hydrogen, alkyl, oxy, alkoxy, or alkanone, or a fifth divalent unit comprising a pendent ultraviolet absorbing group selected from a benzophenone and a benzotriazole. Incorporation of the second, different oligomer may be useful, for example, when the ultraviolet light-absorbing oligomer according to the present disclosure does not comprise any of the third or fifth divalent units. In any of these embodiments, the second, different oligomer can comprise at least one (e.g., at least 2, 3, 4, 5, 6, 7, 8, 9, 10, 15, or at least 20 up to 30, 35, 40, 45, 50, 100, 200, 500, 1000, or up to 1500 or more) second divalent unit, optionally at least one (e.g., at least 2, 3, 4, 5, 6, 7, 8, 9, 10, 15, or at least 20 up to 30, 35, 40, 45, 50, 100, 200, or up to 500 or more) fifth divalent unit, and optionally at least one (e.g., at least 2, 3, 4, 5, 6, 7, 8, 9, 10, 15, or at least 20 up to 30, 35, 40, 45, 50, 100, 200, or up to 500 or more) third divalent unit. Fourth divalent units may also be useful. The fifth, second, third, and fourth divalent units may be as described in any of the embodiments described above for the ultraviolet light-absorbing oligomer. The third or fifth divalent units may be present in the second, different oligomer in a range from 5 to 50 (in some embodiments, 10 to 40 or 10 to 30) percent, based on the total weight of the second oligomer. The second divalent units may be present in the second, different oligomer in a range from 5 to 95 percent, based on the total weight of the second oligomer. In some embodiments, the second divalent unit is present in the second, different oligomer in an amount of up to 90, 80, 75, or 70 percent by weight, based on the total weight of the second oligomer. The mixture of two different ultraviolet-light absorbing oligomers having two different types of pendent UV absorbing groups may be useful to improve performance in some cases. Furthermore, as shown in Int. Pat. Appl. Pub. No. WO2014/100580 (Olson et al.), if an oligomer including a high weight percentage of fourth divalent units results in some non-uniformity in color, haze, or continuity in a film made from the composition, including a second oligomer having a majority of second divalent units in the composition can unexpectedly provide a film having uniform color, haze, and caliper.

In some embodiments, compositions according to the present disclosure include a fluoropolymer, an ultraviolet-light absorbing oligomer, and optionally a second, different oligomer according to any of the aforementioned embodiments. The fluoropolymer is typically a fluorinated thermoplastic obtained by polymerizing one or more types of fully fluorinated or partially fluorinated monomers (e.g., tetrafluoroethylene, vinyl fluoride, vinylidiene fluoride, hexafluoropropylene, pentafluoropropylene, trifluoroethylene, trifluorochloroethylene, and combinations of these in any useful ratio.) Fluoropolymers useful for practicing the present disclosure typically have at least some degree of crystallinity. In some embodiments, fluoropolymers useful for practicing the present disclosure have weight average molecular weights in a range from 30,000 grams per mole to 1,000,000 grams per mole or more. In some embodiments, the weight average molecular weight is at least 40,000 or 50,000 grams per mole up to 500,000, 600,000, 700,000, 800,000, or up to 900,000 grams per mole. Useful fluoropolymers include ethylene-tetrafluoroethylene copolymers (ETFE), tetrafluoroethylene-hexafluoropropylene copolymers (FEP), tetrafluoroethylene-hexafluoropropylene-vinylidene fluoride copolymers (THV), polyvinylidene fluoride (PVDF), blends thereof, and blends of these and other fluoropolymers. Another useful fluoropolymer is a PDVF and hexafluoropropylene (HFP) blend in a variety of useful ratios (e.g., in a range from 50:50 to 95:5 PVDF:HFP, such as 90:10). In some embodiments, the compositions according to the present disclosure include the fluoropolymer in an amount of at least 50, 60, 70, 80, 85, 90, 95, or 96 percent by weight based on the total weight of the composition. In some embodiments, the compositions according to the present disclosure include the fluoropolymer in an amount greater than 95 percent by weight, based on the total weight of the composition. In some embodiments, the compositions according to the present disclosure include the fluoropolymer in an amount of up to 99.5, 99, or 98 percent by weight based on the total weight of the composition.

The composition comprising the fluoropolymer and the oligomer described above can also include non-fluorinated materials. For example, the composition can include poly(methyl methacrylate) (PMMA) polymer or a copolymer of methyl methacrylate and a C₂-C₈ alkyl acrylate or methacrylate. The PMMA polymer or copolymer can have a weight average molecular weight of at least 50,000 grams per mole, 75,000 grams per mole, 100,000 grams per mole, 120,000 grams per mole, 125,000 grams per mole, 150,000 grams per mole, 165,000 grams per mole, or 180,000 grams per mole. The PMMA polymer or copolymer may have a weight average molecular weight of up to 500,000 grams per mole, in some embodiments, up to 400,000 grams per mole, and in some embodiments, up to 250,000 grams per mole. In some embodiments, a blend of polyvinylidene fluoride and poly(methyl methacrylate) can be useful.

In some embodiments, oligomers disclosed herein can be useful in films including a blend of PVDF and PMMA. In these embodiments, it is typically useful for the PMMA to be present in the blend in a range from 10% to 25%, in some embodiments, 15% to 25% or 10% to 20% by weight, based on the total weight of PVDF and PMMA. Films that include much higher amounts of PMMA (e.g., greater than 50% by weight, based on the total weight of PVDF and PMMA) typically have poorer photodurability, higher flammability, and poorer flexibility than films that include PVDF blended with 10% to 25% by weight PMMA. As shown in Examples 15 to 17 of Int. Pat. Appl. No. WO2014/100580 (Olson et al.), when ultraviolet light-absorbing oligomers disclosed herein are used in a film blend of PVDF and PMMA in which the PMMA to be present in the film blend in a range from 10% to 25% by weight, the retention of the ultraviolet light-absorbing oligomers disclosed herein after exposure to ultraviolet light was surprisingly superior to a PVDF film including the oligomers but not including PMMA. Accordingly, the present disclosure provides a composition that includes a blend of a polyvinylidene fluoride and poly(methyl methacrylate) and an ultraviolet light-absorbing oligomer and optionally a second oligomer. When it is said that the poly(methyl methacrylate) is present in the blend in a range from 10% to 25% by weight, based on the total weight of polyvinylidene fluoride and poly(methyl methacrylate), the percentage of poly(methyl methacrylate) in the blend is relative only to the polyvinylidene fluoride and poly(methyl methacrylate), and does not reflect the presence of oligomer. Even when an ultraviolet light-absorbing oligomer disclosed herein includes a second divalent unit derived from methyl methacrylate, the oligomer does not contribute to the percentage of poly(methyl methacrylate).

The composition according to the present disclosure typically includes a blend of the fluoropolymer, the oligomer or oligomers, and any non-fluorinated polymers. By “blend” it is meant that the fluoropolymer and the oligomer according to the present disclosure are not located in separate, distinguishable domains. In other words, the oligomer is typically dispersed throughout the composition; it is not isolated as if in a core-shell polymer particle. Also, by “blend” it should be understood that the fluoropolymer and the ultraviolet light-absorbing oligomer(s) are distinct components. The components of the blend are generally not covalently bonded to each other. Ultraviolet light-absorbing monomers grafted onto a fluoropolymer do not constitute a blend of the fluoropolymer and the oligomer(s) as disclosed herein. In many embodiments, the components of the composition are surprisingly compatible, and the composition appears homogeneous when the components are blended together.

Compositions according to the present disclosure may contain organic solvent. Any solvent that can dissolve the fluoropolymer and oligomer may be useful. The non-volatile components (that is, the components other than solvent) may be present in the solvent at any suitable concentration. For example, the non-volatile components may be present in a range from about 5 percent to about 90 percent by weight, from about 30 percent to about 70 percent by weight, or from about 40 percent to 65 percent by weight, based on the total weight of the composition and solvent). Examples of suitable solvents include aliphatic and alicyclic hydrocarbons (e.g., hexane, heptane, and cyclohexane), aromatic solvents (e.g., benzene, toluene, and xylene), ethers (e.g., diethyl ether, glyme, diglyme, and diisopropyl ether), esters (e.g., ethyl acetate and butyl acetate), alcohols (e.g., ethanol, isopropyl alcohol, and 1-methoxy-2-propanol), and ketones (e.g., acetone, methyl ethyl ketone, and methyl isobutyl ketone). In some embodiments, the solvent comprises at least one of methyl ethyl ketone, acetone, ethyl acetate, 1-methoxy-2-propanol, isopropanol, and toluene. Films of the compositions according to the present disclosure (e.g., including fluoropolymers) may be cast out of solvent.

The advantageous compatibility of the oligomer according to the present disclosure and the fluoropolymer in the compositions disclosed herein allows the compositions to be compounded without organic solvent. Accordingly, in some embodiments, the composition is essentially free of volatile organic solvent. Volatile organic solvents are typically those have a boiling point of up to 150° C. at atmospheric pressure. Examples of these include esters, ketones, and toluene. “Essentially free of volatile organic solvent” can mean that volatile organic solvent may be present (e.g., from a previous synthetic step or in a commercially available monomer) in an amount of up to 2.5 (in some embodiments, up to 2, 1, 0.5, 0.1, 0.05, or 0.01) percent by weight, based on the total weight of the composition. Advantageously, compositions disclosed herein and their films can be made without the expensive manufacturing step of removing organic solvent.

Advantageously, the oligomer and the fluoropolymer can be melt-processed, compounded, mixed, or milled on conventional equipment. Conveniently, uniform masterbatch compositions can be made that include the ultraviolet light-absorbing oligomer at relatively high concentrations in the fluoropolymer. The masterbatch compositions can be extruded (e.g., in a single- or twin-screw extruder) and formed into films. After extrusion, the compositions can also be pelletized or granulated. The masterbatch compositions can then be extrusion compounded with additional fluoropolymer or non-fluorinated polymer (e.g., PMMA) and formed into films.

Other stabilizers may be added to the compositions according to the present disclosure to improve resistance to UV light. Examples of these include hindered amine light stabilizers (HALS) and anti-oxidants. If the third divalent units are not present in the ultraviolet light-absorbing oligomers described above or second, different oligomers in the compositions, conventional HALS may be added to the composition. Some suitable HALS include a tetramethylpiperidine group, in which the nitrogen atoms on the piperidine may be unsubstituted or substituted by alkyl or acyl. Suitable HALS include decanedioic acid, bis (2,2,6,6-tetramethyl-1-(octyloxy)-4-piperidinyl)ester, bis(2,2,6,6-tetramethyl-4-piperidyl)sebacate, 8-acetyl-3-dodecyl-7,7,9,9-tetramethyl-1,3,8-triazaspiro(4,5)-decane-2,5-dione, bis(2,2,6,6-tetramethyl-4-hydroxypiperidine succinate), and bis(N-methyl-2,2,6,6-tetramethyl-4-piperidyl)secacate. Suitable HALS include those available, for example, from BASF under the trade designations “CHIMASSORB”. Exemplary anti-oxidants include those obtained under the trade designations “IRGAFOS 126”, “IRGANOX 1010” and “ULTRANOX 626”, available from BASF, Florham Park, N.J. These stabilizers, if present, can be included in the compositions according to the present disclosure in any effective amount, typically up to 5, 2, to 1 percent by weight based on the total weight of the composition and typically at least 0.1, 0.2, or 0.3 percent by weight. Calcite may also be a useful additive in some compositions, for example, to protect against corrosion of processing equipment not made of corrosion resistant steel.

In some embodiments of the composition according to the present disclosure, the composition can be included in one or more layers of a multilayer film. The multilayer film is any film having more than one layer, typically in the thickness direction of the film. For example, the multilayer film may have at least two or three layers up to 10, 15, or 20 layers. In some embodiments, the composition may be included in a mirror film, which may have a layer (or layers) of the composition according to the present disclosure and a metal layer. In some embodiments, the composition can be included in a multilayer optical film (that is, having an optical layer stack), for example, such as those described in U.S. Pat. App. Pub. Nos. 2009/0283144 (Hebrink et al.) and 2012/0011850 (Hebrink et al.). Multi-layer optical films may have, for example, at least 100, 250, 500, or even at least 1000 optical layers. Such multi-layer optical films can be useful as ultraviolet light-reflective mirrors, visible light-reflective mirrors, infrared light-reflective mirrors, or any combination of these (e.g., broadband reflective mirrors). In some of these embodiments, the multilayer optical film reflects at least a major portion of the average light across the range of wavelengths that corresponds with the absorption bandwidth of a selected photovoltaic cell and does not reflect a major portion of the light that is outside the absorption bandwidth of the photovoltaic cell. In other embodiments, the multilayer optical film may be combined with a metal layer to provide a broadband reflector. In some embodiments, the composition according to the present disclosure may be useful, for example, as a retroreflective sheet.

In view of the advantageous compatibility of the ultraviolet light-absorbing oligomer and the fluoropolymer in the compositions disclosed herein, the present disclosure provides a method of making a composition and a method of making a film. The method of making a composition includes blending the ultraviolet light-absorbing oligomer and optionally the second oligomer with a fluoropolymer to make the composition. The method of making a film includes providing a composition according to the present disclosure, which includes a blend of at least the fluoropolymer, the ultraviolet light-absorbing oligomer, and optionally the second oligomer and extruding the composition into a film. The method may also include blending the composition with additional fluoropolymer or non-fluorinated polymer (e.g., if the composition is a masterbatch composition) before extruding the composition into a film.

In some embodiments, compositions according to the present disclosure are transmissive to both visible and infrared light. The term “transmissive to visible and infrared light” as used herein can mean having an average transmission over the visible and infrared portion of the spectrum of at least about 75% (in some embodiments at least about 80, 85, or 90, 92, 95, 97, or 98%) measured along the normal axis. In some embodiments, the composition has an average transmission over a range of 400 nm to 1400 nm of at least about 75% (in some embodiments at least about 80, 85, 90, 92, 95, 97, or 98%) measured along the normal axis.

The compositions according to the present disclosure can include the ultraviolet light-absorbing oligomer and optionally the second, different oligomer in a range of useful amounts. For example, the ultraviolet light-absorbing oligomer may be present in the composition at up to about 25 percent by weight, based on the total weight of the composition. In some embodiments, the second, different oligomer as described in any of the aforementioned embodiments is present in the composition in an amount of up to ten percent by weight, based on the total weight of the composition. When the ultraviolet light-absorbing oligomer and the second, different oligomer are both present, the two are present in the composition in an amount up to 25 percent combined weight, based on the total weight of the composition. Useful amounts of the ultraviolet light-absorbing oligomer(s) may be in a range from 1 to 25, 2 to 20, 3 to 15, or 4 to 10 percent by weight, based on the total weight of the composition. As shown in the Examples, below, compositions with ultraviolet light-absorbing oligomers in this range are quite effective at absorbing ultraviolet light, and the ultraviolet light protection is maintained even after weathering or exposure to heat and humidity. This is unexpected in view of JP2001/19895, published Jan. 23, 2001, which suggests that polymeric ultraviolet light absorbers are most useful in compositions at 30 to 60 parts per hundred. Useful amounts of the ultraviolet light-absorbing group (in other words, active UVA) may be in a range from 0.5 to 15, 0.5 to 10, 1 to 7.5, or 2 to 5 percent by weight, based on the total weight of the composition.

The advantageous compatibility of the ultraviolet light-absorbing oligomer and the fluoropolymer in the compositions disclosed herein, which allows the compositions to be extrusion compounded, for example, is not found in many compositions including UVAs and fluoropolymers. For example, while a compound represented by formula

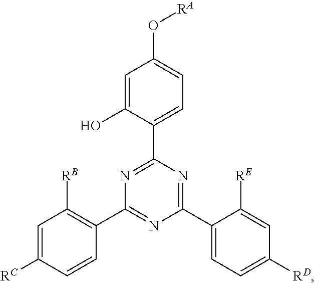

wherein R^{A} is C₁₋₂₀ alkyl or aryl and R^{B}, R^{C}, R^{D}, and R^{E} are hydrogen, C₁₋₅ alkyl, hydroxyl, or aryl are said to be useful UVAs in polymer blends (see, e.g., JP2001/001478, published Jan. 9, 2001), Comparative Example 1, below, shows that and 2-[4-[(2-hydroxy-3-(2′-ethyl)hexyl)oxy]-2-hydroxyphenyl]-4,6-bis(2,4-dimethylphenyl)-1,3,5-triazine when mixed with PVDF and HFP did not provide UV protection after exposure to weathering. Also, when a triazine UV absorber obtained from BASF, Florham Park, N.J., under the trade designation “TINUVIN 1600” was extrusion compounded with PVDF, the resulting strands were very hazy and difficult to pelletize.

Furthermore, while incorporating acryloyl or methacryloyl functional 2-hydroxybenzophenones or 2-hydroxyphenyl-2H-benzotriazoles into high molecular weight PMMA has been proposed, low weathering resistance was observed in comparison to non-covalently attached UVAs (see, U.S. Pat. Appl. Pub. No. 2010/0189983 (Numrich et al.). In contrast the oligomers according to the present disclosure have excellent resistance to weathering, as demonstrated by high retention of percent transmission of visible light and low transmission of ultraviolet light after weathering according to the method described in the Examples, below.

While the retention of the ultraviolet light-absorbing oligomers disclosed herein after exposure to ultraviolet light is generally much superior to the retention of conventional ultraviolet light absorbers after exposure to the same conditions, when the ultraviolet light-absorbing oligomer further includes the third divalent unit having the pendent 2,2,6,6-tetramethylpiperidinyl group and/or when the composition includes a second, different oligomer including the second and third divalent units, the retention of the ultraviolet-light absorbing oligomers after exposure to ultraviolet light may be even better.

Oligomers according to the present disclosure may also be useful, for example, in pressure sensitive adhesives. PSAs are well known to those of ordinary skill in the art to possess properties including the following: (1) aggressive and permanent tack, (2) adherence with no more than finger pressure, (3) sufficient ability to hold onto an adherend, and (4) sufficient cohesive strength to be cleanly removable from the adherend. Materials that have been found to function well as PSAs are polymers designed and formulated to exhibit the requisite viscoelastic properties resulting in a desired balance of tack, peel adhesion, and shear holding power.

One method useful for identifying pressure sensitive adhesives is the Dahlquist criterion. This criterion defines a pressure sensitive adhesive as an adhesive having a 1 second creep compliance of greater than 1×10⁻⁶ cm²/dyne as described in “Handbook of Pressure Sensitive Adhesive Technology”, Donatas Satas (Ed.), 2nd Edition, p. 172, Van Nostrand Reinhold, New York, N.Y., 1989. Alternatively, since modulus is, to a first approximation, the inverse of creep compliance, pressure sensitive adhesives may be defined as adhesives having a storage modulus of less than about 1×10⁶ dynes/cm².

Examples of useful classes PSAs that may include the ultraviolet light-absorbing oligomers according to the present disclosure include acrylic, silicone, polyisobutylene, urea, natural rubber, synthetic rubber such as an ABA triblock copolymer of styrene or substituted styrene as the A blocks and polybutadiene, hydrogenated polybutadiene, polyisoprene, hydrogenated polyisoprene, or a combination thereof as the B block, and combinations of these classes. Some useful commercially available PSAs into which the ultraviolet light-absorbing oligomer according to the present disclosure can be incorporated include UV curable PSAs such as those available from Adhesive Research, Inc., Glen Rock, Pa., under the trade designations “ARclear 90453” and “ARclear 90537” and acrylic optically clear PSAs available, for example, from 3M Company, St. Paul, Minn., under the trade designations “OPTICALLY CLEAR LAMINATING ADHESIVE 8171”, “OPTICALLY CLEAR LAMINATING ADHESIVE 8172”, and “OPTICALLY CLEAR LAMINATING ADHESIVE 8172P”.

In some embodiments, the PSA composition into which the ultraviolet light-absorbing oligomer according to the present disclosure can be incorporated does not flow and has sufficient barrier properties to provide slow or minimal infiltration of oxygen and moisture through the adhesive bond line. Also, the PSA composition may be generally transmissive to visible and infrared light such that it does not interfere with transmission of visible light, for example, through a window film or absorption of visible light, for example, by photovoltaic cells. The PSAs may have an average transmission over the visible portion of the spectrum of at least about 75% (in some embodiments at least about 80, 85, 90, 92, 95, 97, or 98%) measured along the normal axis. In some embodiments, the PSA has an average transmission over a range of 400 nm to 1400 nm of at least about 75% (in some embodiments at least about 80, 85, 90, 92, 95, 97, or 98%) measured along the normal axis.

In some embodiments, useful PSA compositions disclosed herein have a modulus (tensile modulus) up to 50,000 psi (3.4×10⁸ Pa). The tensile modulus can be measured, for example, by a tensile testing instrument such as a testing system available from Instron, Norwood, Mass., under the trade designation “INSTRON 5900”. In some embodiments, the tensile modulus of the PSA is up to 40,000, 30,000, 20,000, or 10,000 psi (2.8×10⁸ Pa, 2.1×10⁸ Pa, 1.4×10⁸ Pa, or 6.9×10⁸ Pa).

In some embodiments, PSAs compositions that include the ultraviolet light-absorbing oligomer according to the present disclosure are acrylic PSAs. As used herein, the term “acrylic” or “acrylate” includes compounds having at least one of acrylic or methacrylic groups. Useful acrylic PSAs can be made, for example, by combining at least two different monomers (second and sixth monomers as described above). Examples of suitable second monomers include 2-methylbutyl acrylate, 2-ethylhexyl acrylate, isooctyl acrylate, lauryl acrylate, n-decyl acrylate, 4-methyl-2-pentyl acrylate, isoamyl acrylate, sec-butyl acrylate, isononyl acrylate, and methacrylates of the foregoing acrylates. Examples of suitable sixth monomers include a (meth)acrylic acid (e.g., acrylic acid, methacrylic acid, itaconic acid, maleic acid, and fumaric acid), a (meth)acrylamide (e.g., acrylamide, methacrylamide, N-ethyl acrylamide, N-hydroxyethyl acrylamide, N-octyl acrylamide, N-t-butyl acrylamide, N,N-dimethyl acrylamide, N,N-diethyl acrylamide, N-ethyl-N-dihydroxyethyl acrylamide, and methacrylamides of the foregoing acrylamides), a (meth)acrylate (e.g., 2-hydroxyethyl acrylate or methacrylate, cyclohexyl acrylate, t-butyl acrylate, isobornyl acrylate, and methacrylates of the foregoing acrylates), N-vinyl pyrrolidone, N-vinyl caprolactam, an alpha-olefin, a vinyl ether, an allyl ether, a styrenic monomer, or a maleate. It can be useful for the pressure sensitive adhesive to include the same second divalent units and optionally the same sixth divalent units as the ultraviolet light-absorbing oligomer described above.

Acrylic PSAs may also be made by including cross-linking agents in the formulation. Examples of cross-linking agents include copolymerizable polyfunctional ethylenically unsaturated monomers (e.g., 1,6-hexanediol diacrylate, trimethylolpropane triacrylate, pentaerythritol tetraacrylate, and 1,2-ethylene glycol diacrylate); ethylenically unsaturated compounds which in the excited state are capable of abstracting hydrogen (e.g., acrylated benzophenones such as described in U.S. Pat. No. 4,737,559 (Kellen et al.), p-acryloxy-benzophenone, which is available from Sartomer Company, Exton, Pa., monomers described in U.S. Pat. No. 5,073,611 (Rehmer et al.) including p-N-(methacryloyl-4-oxapentamethylene)-carbamoyloxybenzophenone, N-(benzoyl-p-phenylene)-N′-(methacryloxymethylene)-carbodiimide, and p-acryloxy-benzophenone); nonionic crosslinking agents which are essentially free of olefinic unsaturation and is capable of reacting with carboxylic acid groups, for example, in the sixth monomer described above (e.g., 1,4-bis(ethyleneiminocarbonylamino)benzene; 4,4-bis(ethyleneiminocarbonylamino)diphenylmethane; 1,8-bis(ethyleneiminocarbonylamino)octane; 1,4-tolylene diisocyanate; 1,6-hexamethylene diisocyanate, N,N′-bis-1,2-propyleneisophthalamide, diepoxides, dianhydrides, bis(amides), and bis(imides)); and nonionic crosslinking agents which are essentially free of olefinic unsaturation, are noncopolymerizable with the first and second monomers, and, in the excited state, are capable of abstracting hydrogen (e.g., 2,4-bis(trichloromethyl)-6-(4-methoxy)phenyl)-s-triazine; 2,4-bis(trichloromethyl)-6-(3,4-dimethoxy)phenyl)-s-triazine; 2,4-bis(trichloromethyl)-6-(3,4,5-trimethoxy)phenyl)-s-triazine; 2,4-bis(trichloromethyl)-6-(2,4-dimethoxy)phenyl)-s-triazine; 2,4-bis(trichloromethyl)-6-(3-methoxy)phenyl)-s-triazine as described in U.S. Pat. No. 4,330,590 (Vesley); 2,4-bis(trichloromethyl)-6-naphthenyl-s-triazine and 2,4-bis(trichloromethyl)-6-(4-methoxy)naphthenyl-s-triazine as described in U.S. Pat. No. 4,329,384 (Vesley)).

Typically, the second monomer is used in an amount of 80-100 parts by weight (pbw) based on a total weight of 100 parts of copolymer, and the sixth monomer is used in an amount of 0-20 pbw based on a total weight of 100 parts of copolymer. The crosslinking agent can be used in an amount of 0.005 to 2 weight percent based on the combined weight of the monomers, for example from about 0.01 to about 0.5 percent by weight or from about 0.05 to 0.15 percent by weight.

The acrylic PSAs useful for practicing the present disclosure can be prepared, for example, in solvent or by a solvent free, bulk, free-radical polymerization process (e.g., using heat, electron-beam radiation, or ultraviolet radiation). Such polymerizations are typically facilitated by a polymerization initiator (e.g., a photoinitiator or a thermal initiator). Examples of suitable polymerization initiators include an of those described above for the preparation of the ultraviolet light-absorbing oligomer. The polymerization initiator is used in an amount effective to facilitate polymerization of the monomers (e.g., 0.1 part to about 5.0 parts or 0.2 part to about 1.0 part by weight, based on 100 parts of the total monomer content).

If a photocrosslinking agent is used, the coated adhesive can be exposed to ultraviolet radiation having a wavelength of about 250 nm to about 400 nm. The radiant energy in this range of wavelength required to crosslink the adhesive is about 100 millijoules/cm² to about 1,500 millijoules/cm2, or more specifically, about 200 millijoules/cm² to about 800 millijoules/cm².

A useful solvent-free polymerization method is disclosed in U.S. Pat. No. 4,379,201 (Heilmann et al.). Initially, a mixture of second and sixth monomers can be polymerized with a portion of a photoinitiator by exposing the mixture to UV radiation in an inert environment for a time sufficient to form a coatable base syrup, and subsequently adding a crosslinking agent and the remainder of the photoinitiator. This final syrup containing a crosslinking agent (e.g., which may have a Brookfield viscosity of about 100 centipoise to about 6000 centipoise at 23° C., as measured with a No. 4 LTV spindle, at 60 revolutions per minute) can then be coated onto a substrate, for example, a polymeric film substrate. Once the syrup is coated onto the substrate, for example, the polymeric film substrate, further polymerization and crosslinking can be carried out in an inert environment (e.g., nitrogen, carbon dioxide, helium, and argon, which exclude oxygen). A sufficiently inert atmosphere can be achieved by covering a layer of the photoactive syrup with a polymeric film, such as silicone-treated PET film, that is transparent to UV radiation or e-beam and irradiating through the film in air.

PSAs generally include high molecular weight polymers. In some embodiments, the acrylic polymer in the pressure sensitive adhesive in the composition according to the present disclosure has a number average molecular weight of at least 300,000 grams per mole. Number average molecular weights lower than 300,000 grams per mole may produce PSAs with low durability. In some embodiments, the number average molecular weight of the PSA is in the range from 300,000 to 3 million, 400,000 to 2 million, 500,000 to 2 million, or 300,000 to 1 million grams per mole. Accordingly, in some embodiments, the ultraviolet light-absorbing oligomer has a number average molecular weight of up to one half the number average molecular weight of the pressure sensitive adhesive. In some embodiments, the ultraviolet light-absorbing oligomer has a number average molecular weight of up to one-third, one-fifth, or one-tenth the number average molecular weight of the pressure sensitive adhesive.

Compositions according to the present disclosure may be useful for a variety of outdoor applications. For example, the compositions according to the present disclosure may be useful, for example, for top layers of traffic or other signs, other graphic films (e.g., for building or automotive exteriors), roofing materials or other architectural films, or window films or as a PSA layer for any of these films.

Compositions according to the present disclosure are useful, for example, for encapsulating solar devices. In some embodiments, the composition (e.g., in the form of a film or a pressure sensitive adhesive) is disposed on, above, or around a photovoltaic cell. Accordingly, the present disclosure provides a photovoltaic device including the composition disclosed herein in which the composition (e.g., in the form of a film) is used as a top sheet for the photovoltaic device. Photovoltaic devices include photovoltaic cells that have been developed with a variety of materials each having a unique absorption spectrum that converts solar energy into electricity. Each type of semiconductor material has a characteristic band gap energy which causes it to absorb light most efficiently at certain wavelengths of light, or more precisely, to absorb electromagnetic radiation over a portion of the solar spectrum. The compositions according to the present disclosure typically do not interfere with absorption of visible and infrared light, for example, by photovoltaic cells. In some embodiments, the composition has an average transmission over a range wavelengths of light that are useful to a photovoltaic cell of at least about 75% (in some embodiments at least about 80, 85, 90, 92, 95, 97, or 98%) measured along the normal axis. Examples of materials used to make solar cells and their solar light absorption band-edge wavelengths include: crystalline silicon single junction (about 400 nm to about 1150 nm), amorphous silicon single junction (about 300 nm to about 720 nm), ribbon silicon (about 350 nm to about 1150 nm), CIS (Copper Indium Selenide) (about 400 nm to about 1300 nm), CIGS (Copper Indium Gallium di-Selenide) (about 350 nm to about 1100 nm), CdTe (about 400 nm to about 895 nm), GaAs multi-junction (about 350 nm to about 1750 nm). The shorter wavelength left absorption band edge of these semiconductor materials is typically between 300 nm and 400 nm. Organic photovoltaic cells may also be useful. One skilled in the art understands that new materials are being developed for more efficient solar cells having their own unique longer wavelength absorption band-edge. In some embodiments, the photovoltaic device including the composition according to the present disclosure includes a CIGS cell. In some embodiments, the photovoltaic device to which the assembly is applied comprises a flexible film substrate.

A composition according to the present disclosure (e.g., in the form of a film) can be used as a substrate for a barrier stack (see, e.g., U.S. Pat. Appl. Pub. No. 2012/0227809 (Bharti et al.) or can be attached to a barrier stack using an optically clear adhesive such as a pressure sensitive adhesive (PSA) (see, e.g., U.S. Pat. Appl. Pub. No. 2012/0003451 (Weigel et al.). The PSA useful for attaching a top sheet to a barrier stack may include the ultraviolet light-absorbing oligomer disclosed herein and may have any of the features described above. In some embodiments, the top sheet and barrier film assembly is attached to the photovoltaic cell with an encapsulant. Although other encapsulants may be useful, in some embodiments, the encapsulant is ethylene vinylacetate.

### Some Embodiments of the Disclosure

In a first embodiment, the present disclosure provides a composition comprising a blend of a fluoropolymer and an ultraviolet light-absorbing oligomer, wherein the ultraviolet light-absorbing oligomer comprises:

a first divalent unit represented by formula:

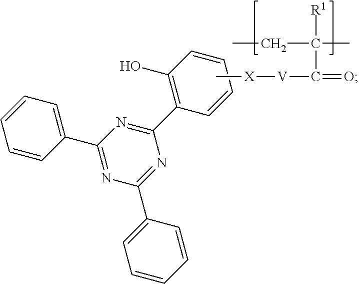

and

a second divalent unit represented by formula:

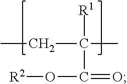

wherein
each R¹ is independently hydrogen or methyl;
V is O or NH;
X is a bond, alkylene, or alkyleneoxy, wherein the alkylene or alkyleneoxy have from 1 to 10 carbon atoms and are optionally interrupted by one or more —O— groups and optionally substituted by a hydroxyl group; and
R² is alkyl having from 1 to 4 carbon atoms.

In a second embodiment, the present disclosure provides the composition of the first embodiment, wherein the ultraviolet light-absorbing oligomer further comprises a third divalent unit represented by formula:

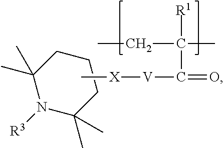

wherein
R¹ is independently hydrogen or methyl;
X is a bond, alkylene, or alkyleneoxy, wherein the alkylene or alkyleneoxy have from 1 to 10 carbon atoms and are optionally interrupted by one or more —O— groups and optionally substituted by a hydroxyl group;
V is O or NH; and
R³ is hydrogen, alkyl, oxy, alkoxy, or alkanone.

In a third embodiment, the present disclosure provides the composition the second embodiment, wherein X is a bond.

In a fourth embodiment, the present disclosure provides the composition of any one of the first to third embodiments, wherein the ultraviolet light-absorbing oligomer further comprises a fifth divalent unit represented by formula:

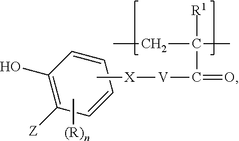

wherein
R¹ is independently hydrogen or methyl;
V is O or NH;
X is a bond, alkylene, or alkyleneoxy, wherein the alkylene or alkyleneoxy have from 1 to 10 carbon atoms and are optionally interrupted by one or more —O— groups and optionally substituted by a hydroxyl group;
R is alkyl having from one to four carbon atoms;
n is 0 or 1; and
Z is a benzoyl group optionally substituted by hydroxyl, alkyl, halogen, or hydroxyl or a 2H-benzotriazol-2-yl group optionally substituted by one or more halogens.

In a fifth embodiment, the present disclosure provides the composition of the fourth embodiment, wherein Z is a 2H-benzotriazol-2-yl group optionally substituted by one or more halogens.

In a sixth embodiment, the present disclosure provides the composition of any one of the first to fifth embodiments, wherein the ultraviolet light-absorbing oligomer further comprises a fourth divalent unit represented by formula:

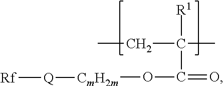

wherein
Rf represents a fluoroalkyl group having from 1 to 8 carbon atoms optionally interrupted by one —O— group, or Rf represents a polyfluoropolyether group;
R¹ is independently hydrogen or methyl;
Q is a bond, —SO₂—N(R⁵)—, or —C(O)—N(R⁵)—, wherein R is alkyl having from 1 to 4 carbon atoms or hydrogen; and
m is an integer from 0 to 10.

In a seventh embodiment, the present disclosure provides the composition any one of the first to sixth embodiments, wherein the ultraviolet light-absorbing oligomer is in the composition in an amount ranging from 1 percent to 25 percent by weight, based on the total weight of the composition.

In an eighth embodiment, the present disclosure provides the composition of any one of the first to seventh embodiments, wherein in the second divalent unit, R¹ and R² are both methyl.

In a ninth embodiment, the present disclosure provides the composition of any one of the first to eighth embodiments, further comprising a second, different oligomer comprising the second divalent units and at least one of:

a third divalent unit comprising a pendent 2,2,6,6-tetramethylpiperidinyl group, wherein the nitrogen of the pendent 2,2,6,6-tetramethylpiperidinyl group is substituted by hydrogen, alkyl, alkoxy, or alkanone; or

a fifth divalent unit comprising a pendent ultraviolet absorbing group selected from a benzophenone and a benzotriazole.

In a tenth embodiment, the present disclosure provides the composition of the ninth embodiment, wherein the second, different oligomer has a number average molecular weight of less than 20,000 grams per mole and wherein R¹ and R² are both methyl.

In an eleventh embodiment, the present disclosure provides the composition of the ninth or tenth embodiment, wherein the second, different oligomer is present in the composition in an amount of up to ten percent by weight, based on the total weight of the composition.

In a twelfth embodiment, the present disclosure the composition of the eleventh embodiment, wherein the 2,2,6,6-tetramethylpiperidinyl group, benzophenone group, or benzotriazole group may be present in the composition in an amount of up to 5 percent by weight, based on the total weight of the composition.

In a thirteenth embodiment, the present disclosure provides the composition of any one of the ninth to twelfth embodiment, wherein the ultraviolet light-absorbing oligomer and the second, different oligomer are present in the composition in an amount of up to 25 percent by weight, based on the total weight of the composition.

In a fourteenth embodiment, the present disclosure provides the composition of any one of the first to thirteenth embodiments, wherein the blend further comprises poly(methyl methacrylate).

In a fifteenth embodiment, the present disclosure provides the composition of the fourteenth embodiment, wherein the fluroropolymer comprises polyvinylidine fluoride, and wherein poly(methyl methacrylate) is present in the composition in an amount from ten percent to 25 percent by weight, based on the total weight of the polyvinylidene fluoride and poly(methyl methacrylate).

In a sixteenth embodiment, the present disclosure provides the composition of the fourteenth or fifteenth embodiment, wherein the poly(methyl methacrylate) has a number average molecular weight of at least 100,000 grams per mole.

In a seventeenth embodiment, the present disclosure provides the composition of any one of the first to sixteenth embodiments, wherein the fluoropolymer is present in the blend in an amount of at least 70 percent by weight, based on the total weight of the composition.

In an eighteenth embodiment, the present disclosure provides the composition of any one of the first to seventeenth embodiments, wherein the fluoropolymer is present in the blend in an amount of at least 90 percent by weight, based on the total weight of the composition.

In a nineteenth embodiment, the present disclosure provides the composition of any one of the first to eighteenth embodiments, wherein the first divalent unit is in the composition in an amount ranging from 0.5 weight percent to 5 weight percent, based on the total weight of the composition.

In a twentieth embodiment, the present disclosure provides the composition of any one of the first to nineteenth embodiments, further comprising a hindered amine light stabilizer.

In a twenty-first embodiment, the present disclosure provides the composition of any one of the first to twentieth embodiments, wherein the composition is in the form of a film.

In a twenty-second embodiment, the present disclosure provides the composition of the twenty-first embodiment, wherein the composition is an extruded film.

In a twenty-third embodiment, the present disclosure provides the composition of any one of the first to twenty-second embodiments, wherein the composition is essentially free of volatile organic solvent.

In a twenty-fourth embodiment, the present disclosure provides the composition of any one of the first to twenty-third embodiments, wherein the ultraviolet light-absorbing oligomer has a number average molecular weight of less than 20,000 grams per mole and wherein R¹ and R² are both methyl.

In a twenty-fifth embodiment, the present disclosure provides the composition of any one of the first to twenty-fourth embodiments, wherein the fluoropolymer is selected from the group consisting of ethylene-tetrafluoroethylene copolymer, a tetrafluoroethylene-hexafluoropropylene copolymer, a tetrafluoroethylene-hexafluoropropylene-vinylidene fluoride copolymer, or polyvinylidene fluoride.

In a twenty-sixth embodiment, the present disclosure provides the composition of any one of the first to twenty-fifth embodiments, wherein the film is a multilayer film.

In a twenty-seventh embodiment, the present disclosure provides the composition of the twenty-sixth embodiment, wherein the film is a multilayer optical film.

In a twenty-eighth embodiment, the present disclosure provides a photovoltaic device comprising the composition of any one of the first to twenty-seventh embodiments.

In a twenty-ninth embodiment, the present disclosure provides a graphic film comprising the composition of any one of the first to twenty-seventh embodiments.

In a thirtieth embodiment, the present disclosure provides an architectural film comprising the composition of any one of the first to twenty-seventh embodiments.

In a thirty-first embodiment, the present disclosure provides a window film comprising the composition of any one of the first to twenty-seventh embodiments.

In a thirty-second embodiment, the present disclosure provides a vehicle wrap comprising the composition of any one of the first to twenty-seventh embodiments.

In a thirty-third embodiment, the present disclosure provides a method of making the composition of any one of the first to twenty-seventh embodiments, the method comprising:

combining the fluoropolymer, the ultraviolet light-absorbing oligomer, and optionally the second, different oligomer to form the blend; and

extruding the blend into a film.

In a thirty-fourth embodiment, the present disclosure provides an ultraviolet light-absorbing oligomer comprising:

a first divalent unit represented by formula:

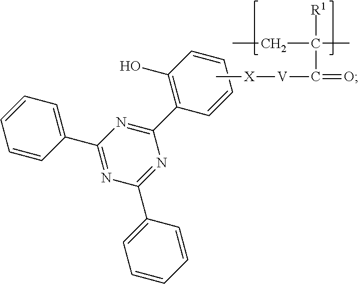

and

a second divalent unit represented by formula:

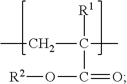

wherein
each R¹ is independently hydrogen or methyl;
V is O or NH;
X is a bond, alkylene, or alkyleneoxy, wherein the alkylene or alkyleneoxy have from 1 to 10 carbon atoms and are optionally interrupted by one or more —O— groups and optionally substituted by a hydroxyl group; and
R² is alkyl having from 1 to 22 carbon atoms.

In a thirty-fifth embodiment, the present disclosure provides the ultraviolet light-absorbing oligomer of the thirty-fourth embodiment, wherein the ultraviolet light-absorbing oligomer further comprises a third divalent unit represented by formula:

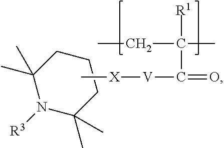

wherein
R¹ is independently hydrogen or methyl;
X is a bond, alkylene, or alkyleneoxy, wherein the alkylene or alkyleneoxy have from 1 to 10 carbon atoms and are optionally interrupted by one or more —O— groups and optionally substituted by a hydroxyl group;
V is O or NH; and
R³ is hydrogen, alkyl, oxy, alkoxy, or alkanone.

In a thirty-sixth embodiment, the present disclosure provides the ultraviolet light-absorbing oligomer the thirty-fourth or thirty-fifth embodiment, wherein X is a bond.

In a thirty-seventh embodiment, the present disclosure provides the ultraviolet light-absorbing oligomer of any one of the thirty-fourth to thirty-sixth embodiments, wherein the ultraviolet light-absorbing oligomer further comprises a fifth divalent unit represented by formula:

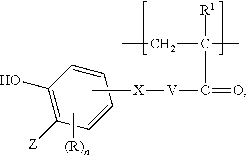

wherein
R¹ is independently hydrogen or methyl;
V is O or NH;
X is a bond, alkylene, or alkyleneoxy, wherein the alkylene or alkyleneoxy have from 1 to 10 carbon atoms and are optionally interrupted by one or more —O— groups and optionally substituted by a hydroxyl group;
R is alkyl having from one to four carbon atoms;
n is 0 or 1; and
Z is a benzoyl group optionally substituted by hydroxyl, alkyl, halogen, or hydroxyl or a 2H-benzotriazol-2-yl group optionally substituted by one or more halogens.

In a thirty-eighth embodiment, the present disclosure provides the composition of the thirty-seventh embodiment, wherein Z is a 2H-benzotriazol-2-yl group optionally substituted by one or more halogens.

In a thirty-ninth embodiment, the present disclosure provides the composition of any one of the thirty-fourth to thirty-eighth embodiments, wherein the ultraviolet light-absorbing oligomer further comprises a fourth divalent unit represented by formula:

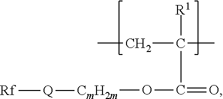

wherein
Rf represents a fluoroalkyl group having from 1 to 8 carbon atoms optionally interrupted by one —O— group, or Rf represents a polyfluoropolyether group;
R¹ is independently hydrogen or methyl;
Q is a bond, —SO₂—N(R⁵)—, or —C(O)—N(R⁵)—, wherein R is alkyl having from 1 to 4 carbon atoms or hydrogen; and
m is an integer from 0 to 10.

In the fortieth embodiment, the present disclosure provides the ultraviolet light-absorbing oligomer of any one of the thirty-fourth to thirty-ninth embodiments, wherein the ultraviolet light-absorbing oligomer further comprises a sixth divalent unit comprising a pendent carboxylic acid, hydroxyl, aminocarbonyl, alkylaminocarbonyl, or dialkylaminocarbonyl group, wherein the alkyl in the alkylaminocarbonyl or dialkylaminocarbonyl is optionally substituted by hydroxyl.

In a forty-first embodiment, the present disclosure provides a composition comprising a blend of a fluoropolymer and the ultraviolet light-absorbing oligomer of any one of the thirty-fourth to fortieth embodiments.

In a forty-second embodiment, the present disclosure provides the composition of the forty-first embodiment, wherein R² is alkyl having 1 to 4 carbon atoms.

In a forty-third embodiment, the present disclosure provides a pressure sensitive adhesive comprising the ultraviolet light-absorbing oligomer of any one of the thirty-fourth to fortieth embodiments.

In a forty-fourth embodiment, the present disclosure provides the pressure sensitive adhesive of the forty-third embodiment, wherein R² is alkyl having 4 to 22 carbon atoms.

In a forty-fifth embodiment, the present disclosure provides the pressure sensitive adhesive of the forty-third or forty-fourth embodiment, wherein the pressure sensitive adhesive comprises at least one of an acrylate, silicone, polyisobutylene, urea, natural rubber, or an ABA triblock copolymer of styrene and polybutadiene, hydrogenated polybutadiene, polyisoprene, hydrogenated polyisoprene, or a combination thereof.

In a forty-sixth embodiment, the present disclosure provides the pressure sensitive adhesive of the forty-third or forty-fourth embodiment, wherein the pressure sensitive adhesive is an acrylic pressure sensitive adhesive.

In a forty-seventh embodiment, the present disclosure provides the pressure sensitive adhesive of the forty-sixth embodiment, wherein the pressure sensitive adhesive comprises the second divalent unit, and wherein R² is alkyl having 4 to 22 carbon atoms.

In the forty-eighth embodiment, the present disclosure provides the pressure sensitive adhesive of any one of the fourth-third to forty-seventh embodiments, wherein the ultraviolet light-absorbing oligomer further comprises a sixth divalent unit comprising a pendent carboxylic acid, hydroxyl, aminocarbonyl, alkylaminocarbonyl, or dialkylaminocarbonyl group, wherein the alkyl in the alkylaminocarbonyl or dialkylaminocarbonyl is optionally substituted by hydroxyl.

In the forty-ninth embodiment, the present disclosure provides the pressure sensitive adhesive of any one of the fourth-third to forty-eighth embodiments, wherein R² is alkyl having 8 carbon atoms.

In a fiftieth embodiment, the present disclosure provides the pressure sensitive adhesive of any one of the fourth-third to forty-ninth embodiments, wherein the ultraviolet light-absorbing oligomer is in the pressure sensitive adhesive in an amount ranging from 1 percent to 25 percent by weight, based on the total weight of the pressure sensitive adhesive.

In a fifty-first embodiment, the present disclosure provides the pressure sensitive adhesive of any one of the fourth-third to fiftieth embodiments, further comprising a second, different oligomer comprising the second divalent units and at least one of:

a third divalent unit comprising a pendent 2,2,6,6-tetramethylpiperidinyl group, wherein the nitrogen of the pendent 2,2,6,6-tetramethylpiperidinyl group is substituted by hydrogen, alkyl, alkoxy, or alkanone; or

a fifth divalent unit comprising a pendent ultraviolet absorbing group selected from a benzophenone and a benzotriazole.

In a fifty-second embodiment, the present disclosure provides the pressure sensitive adhesive of the fifty-first embodiment, wherein the second, different oligomer is present in the composition in an amount of up to ten percent by weight, based on the total weight of the composition.

In a fifty-third embodiment, the present disclosure provides the pressure sensitive adhesive of the fifty-first or fifty-second embodiment, wherein the 2,2,6,6-tetramethylpiperidinyl group, benzophenone group, or benzotriazole group is present in the pressure sensitive adhesive in an amount of up to 5 percent by weight, based on the total weight of the composition.

In a fifty-fourth embodiment, the present disclosure provides the pressure sensitive adhesive of any one of the fifty-first to fifty-third embodiments, wherein the ultraviolet light-absorbing oligomer and the second, different oligomer are present in the pressure sensitive adhesive in an amount of up to 25 percent by weight, based on the total weight of the composition.

In a fifty-fifth embodiment, the present disclosure provides the pressure sensitive adhesive of any one of the forty-third to fifty-fourth embodiments, further comprising a hindered amine light stabilizer.

In a fifty-sixth embodiment, the present disclosure provides a photovoltaic device comprising the pressure sensitive adhesive of any one of the forty-third to fifty-fifth embodiments.

In a fifty-seventh embodiment, the present disclosure provides an article wherein the pressure sensitive adhesive of any one of the forty-third to fifty-sixth embodiments is disposed on a film.

In a fifty-eighth embodiment, the present disclosure provides the article of the fifty-seventh embodiment, wherein the film is at least one of a graphic film, an architectural film, a window film, or a vehicle wrap.

Embodiments of the methods disclosed herein are further illustrated by the following examples, but the particular materials and amounts thereof recited in these examples, as well as other conditions and details, should not be construed to unduly limit this invention.

### EXAMPLES

### Molecular Weight Determination

In the following oligomer examples, the molecular weight was determined by comparison to linear polystyrene polymer standards using gel permeation chromatography (GPC). The GPC measurements were carried out on a Waters Alliance 2695 system (obtained from Waters Corporation, Milford, Mass.) using four 300 millimeter (mm) by 7.8 mm linear columns of 5 micrometer styrene divinylbenzene copolymer particles (obtained from Polymer Laboratories, Shropshire, UK, under the trade designation “PLGEL”) with pore sizes of 10,000, 1000, 500, and 100 angstroms. A refractive index detector from Waters Corporation (model 410) was used at 40° C. A 50-milligram (mg) sample of oligomer in ethyl acetate was diluted with 10 milliliters (mL) of tetrahydrofuran (inhibited with 250 ppm of BHT) and filtered through a 0.45 micrometer syringe filter. A sample volume of 100 microliters was injected onto the column, and the column temperature was 40° C. A flow rate of 1 mL/minute was used, and the mobile phase was tetrahydrofuran. Molecular weight calibration was performed using narrow dispersity polystyrene standards with peak average molecular weights ranging from 3.8×10⁵ grams per mole to 580 grams per mole. Calibration and molecular weight distribution calculations were performed using suitable GPC software using a third order polynomial fit for the molecular weight calibration curve. Each reported result is an average of duplicate injections.

### Glass Transition Temperature

For the following oligomer examples, the glass transition temperatures were measured by Differential Scanning calorimetry (DSC) using Q2000 Differential Scanning calorimeter obtained from TA Instruments, New Castle, Del. Glass transition temperature was determined using Modulated DSC with a modulation amplitude off 1° C. per minute and a ramp rate of 3° C. per minute.

### Accelerated Ultraviolet Light Exposure

Films were exposed in a weathering device according to a high-irradiance version of ASTM G155 Cycle 1 run at slightly higher temperature. Radiation from the xenon arc source was appropriately filtered so as to provide an excellent match to the ultraviolet portion of the solar spectrum. Samples were tested prior to any exposure to these accelerated weathering conditions, and then removed at total UV dosage intervals of about 373 MJ/m² for evaluation. The number of these dosage intervals to which the Examples were exposed are specified below.

### Preparative Example 1

### 2-[4-(4,6-Diphenyl)-[1,3,5]triazin-2-yl]-3-hydroxy-phenoxy}-ethyl prop-2-enoate

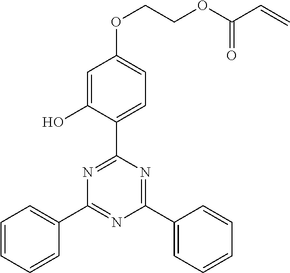 [SMILES: C=CC(=O)OCCOc1ccc(-c2nc(-c3ccccc3)nc(-c3ccccc3)n2)c(O)c1]

### Part A

A two liter 3-neck round bottom flask was equipped with a temperature probe, condenser and mechanical stirrer. The flask was charged with 400 grams (1.17 moles) of 4-(4,6-diphenyl-1,3,5-triazin-2-yl)benzene-1,3-diol, 115.5 grams (1.31 moles) of ethylene carbonate, 16.7 grams (0.085 moles) tetraethylammonium bromide and 440 grams of dimethyl formamide (DMF). The batch was heated to 150° C. and maintained at that temperature for five hours. The evolution of CO₂ from the batch was observed. After five hours, 10 grams additional ethylene were added. The batch was heated at 150° C. for three hours, and then 15 grams additional ethylene carbonate and 2 grams additional tetraethylammonium bromide were added. The batch was heated at 150° C. for three more hours, after which time no more starting material was observed by thin layer chromatography.

The batch was allowed to cool to 80° C., and 730 grams of isopropanol (IPA) was added. The mixture was thick, and a mixture of 50/50 IPA/water was added to improve stirring. The solid product was then collected by filtration onto a Buchner funnel. The solid product was taken up into 2500 grams of DMF, heated at reflux, cooled to room temperature, and collected by filtration onto a Buchner funnel. The product was air-dried to give 373 grams (83%) of an off-white solid product 2-(4,6-diphenyl-1,3,5-triazin-2-yl)-5-(2-hydroxyethoxy)phenol.

### Part B

A two liter 3-neck round bottom flask was equipped with a temperature probe, Dean-Stark trap with condenser, and mechanical stirrer. The flask was charged with 150 grams (0.389 moles) of 2-(4,6-diphenyl-1,3,5-triazin-2-yl)-5-(2-hydroxyethoxy)phenol, prepared in Part A, 790 grams of toluene, 0.24 grams of 4-methoxyphenol (MEHQ) inhibitor, 0.38 grams of phenothiazine inhibitor, 8.5 grams of p-toluene sulfonic acid, and 30.8 grams (0.43 mole) of acrylic acid. The batch was heated with medium agitation at reflux (about 115° C.) for six hours, and the azeotroped water was collected in the Dean-Stark trap. After five hours, five grams additional acrylic acid was added, and the batch was heated for three more hours. Analysis by thin layer chromatography eluting with 50/50 ethyl acetate/hexanes showed the batch had no residual starting material.

The batch was allowed to cool to 80° C., and 65 grams of triethyl amine was added. The batch was heated at reflux at atmospheric pressure to remove most of the toluene. The pot temperature was 120° C., and about 650 grams of toluene were collected. The batch was allowed to cool to 75° C., and 500 grams IPA were added. The mixture was heated at reflux (about 82° C.) to azetrope off the toluene and IPA. About 500 grams of solvent were collected. The reaction mixture was cooled to about 20° C. with an ice bath, and 500 grams of IPA were added with stirring. The precipitated product was collected by filtration on a Buchner funnel. The solid was taken back up in a mixture of 700 grams water and 700 grams IPA, and the mixture was stirred well and filtered. The product was air-dried to give 161.8 grams (95%) of the light yellow solid product, mp=125° C.-127° C.

To further purify, about 90 grams of the light yellow solid was combined with 1200 grams MEK and heated to 40° C. Five grams of charcoal was added, and the mixture was stirred well and filtered through a bed of filter aid. The solvent was removed using a rotary evaporator, and then 400 grams IPA was added. The mixture was stirred well, and the solid product 2-[4-(4,6-diphenyl)-[1,3,5]triazin-2-yl]-3-hydroxy-phenoxy}-ethyl prop-2-enoate, was collected by filtration. mp=126° C. to 128° C. The structure was confirmed by ¹H NMR spectroscopy.

### Preparative Example 2

### 2-{4-[4,6-Bis-(2,4-dimethyl-phenyl)-[1,3,5]triazin-2-yl]-3-hydroxy-phenoxy}-ethyl acrylate ester

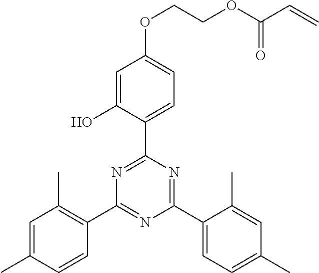 [SMILES: C=CC(=O)OCCOc1ccc(-c2nc(-c3ccc(C)cc3C)nc(-c3ccc(C)cc3C)n2)c(O)c1]

### Part A

A three liter 3-neck round bottom flask was equipped with a temperature probe, condenser and mechanical stirrer. The flask was charged with 500 grams (1.26 moles) of 2,4-di-(2,4-dimethylphenyl)-6-(2,4-dihydroxyphenyl)-triazine, 124 grams (1.4 moles) of ethylene carbonate, 18 grams (0.085 moles) tetraethylammonium bromide and 475 grams of dimethyl formamide. The batch was heated to 150° C. and maintained at that temperature for five hours. The evolution of CO₂ from the batch was observed. After five hours, 15 grams additional ethylene carbonate and 2 grams additional tetraethylammonium bromide were added. The batch was heated at 150° C. for three hours, and then 15 grams additional ethylene carbonate and 2 grams additional tetraethylammonium bromide were added. The batch was heated at 150° C. for three more hours, after which time no more starting material was observed by thin layer chromatography.

The batch was allowed to cool to 80° C., and 1360 grams of isopropanol (IPA) was added with good agitation. The mixture was cooled to room temperature, and the solid product was collected by filtration onto a Buchner funnel. The solid product was taken up into 1000 grams each of water and IPA, stirred well, and collected by filtration onto a Buchner funnel. The product was air-dried to give 540 grams (96%) of an off-white solid product, 2-[4,6-bis-(2,4-dimethylphenyl)-[1,3,5]triazin-2-yl]-5-(2-hydroxyethoxy)phenol, mp=172° C.-173° C. The product was used without further purification.

### Part B

A two liter 3-neck round bottom flask was equipped with a temperature probe, Dean-Stark trap with condenser, and mechanical stirrer. The flask was charged with 170 grams (0.385 moles) of 2-[4,6-bis-(2,4-dimethylphenyl)-[1,3,5]triazin-2-yl]-5-(2-hydroxyethoxy)phenol, prepared in Part A, 780 grams of toluene, 0.24 grams of 4-methoxyphenol (MEHQ) inhibitor, 0.38 grams of phenothiazine inhibitor, 8.5 grams of p-toluene sulfonic acid, and 30.5 grams (0.42 moles) of acrylic acid. The batch was heated with medium agitation at reflux (about 115° C.) for six hours, and the azeotroped water can collected in the Dean-Stark trap. After five hours, five grams additional acrylic acid was added, and the batch was heated for three more hours. Analysis by thin layer chromatography showed the batch had no residual starting material.

The batch was allowed to cool to 80° C., and a pre-mix of 25 grams sodium carbonate in 300 grams water was added. The reaction mixture was cooled to about 10° C. with an ice bath, and the precipitated product was collected by filtration on a Buchner funnel. The solid was taken back up in a mixture of 800 grams water and 200 grams IPA, and the mixture was stirred well and filtered. The product was air-dried to give 182 grams (96%) of the off-white solid product, 2-{4-[4,6-bis-(2,4-dimethyl-phenyl)-[1,3,5]triazin-2-yl]-3-hydroxyphenoxy}ethyl acrylate ester, mp=126° C.-128° C. The structure was confirmed by ¹H NMR spectroscopy.

### Oligomer Example 1

### Random Copolymer of 80% by weight Methyl Methacrylate and 20% Preparative Example 1

Twenty grams of Preparative Example 1, 80 g methyl methacrylate (obtained from Alfa Aesar, Ward Hill, Mass.), and 400 g of ethyl acetate were added to a one-liter flask fitted with a thermocouple, overhead stirrer, and a reflux condenser under positive nitrogen flow. After the addition of materials, the flask was maintained under positive nitrogen pressure. The set point on the controller for the thermocouple (obtained from J-Kem, St. Louis, Mo.) was set to 70° C., and 2.8 g of 2,2′-azobis(2-methylbutyronitrile) (obtained from E.I. du Pont de Nemours and Company, Wilmington, Del., under the trade designation “VAZO 67”) were added. The batch was allowed to stand for 15 minutes. The set point was raised to 74° C., and the timer was set for 18 hours. After the time had expired, the contents of the flask were poured out into an aluminum tray and air-dried overnight. The next day, the product was dried in an oven at 100° C. for 18 hours and then one hour at 140° C. to give 98 g of oligomer. One glass transition temperature was observed at 107.9° C. using DSC according to the method described above with a scan from −100° C. to 150° C. The molecular weight of the oligomer was determined by GPC (THF, EMD Omnisolve, 2c PL-Gel-2 300×7.5 mm, polystyrene standard): Mw=17301, Mn=3608, and a polydispersity of 4.8.

### Oligomer Example 2

### Random Copolymer of 75% by Weight Methyl Methacrylate, 10% Preparative Example 1, 10% 2-[2-Hydroxy-5-[2-(methacryloyloxy)-ethyl]phenyl]-2H-benzotriazole, and 5% 2,2,6,6-Tetramethyl-4-piperidyl Methacrylate

2-[2-Hydroxy-5-[2-(methacryloyloxy)-ethyl]phenyl]-2H-benzotriazole was obtained from TCI America, Portland, Oreg.

Oligomer Example 2 was prepared according to the method of Oligomer Example 1, with the modification that 10 g of Preparative Example 1, 10 g of 2-[2-hydroxy-5-[2-(methacryloyloxy)-ethyl]phenyl]-2H-benzotriazole, 75 g of methyl methacrylate and 200 grams ethyl acetate were initially added to the flask. After the solid was collected and air-dried overnight, the product was dried in an oven at 100° C. for 18 hours and then one hour at 150° C. to give 101 g of oligomer. One glass transition temperature was observed at 106.3° C. using DSC according to the method described above with a scan from −100° C. to 150° C.

### Oligomer Example 3

### Random Copolymer of 80% by Weight Isooctyl Acrylate, 20% by Weight Preparative Example 1

Forty grams of isooctyl acrylate (obtained from TCI America) was mixed with 10 g of Preparative Example 1, 1 g of 2,2′-azobis(2-methylbutyronitrile) (obtained from E.I. du Pont de Nemours and Company, Wilmington, Del., under the trade designation “VAZO 67”), and 100 g of ethyl acetate in a one-liter flask fitted with a thermocouple, overhead stirrer, and a reflux condenser under positive nitrogen flow. After the addition of materials was completed, the flask was maintained under positive nitrogen pressure. The material was heated at 74° C. for 1 hour and then another 1 g of 2,2′-azobis(2-methylbutyronitrile) was added. The material was heated at 74° C. for 18 hours. The contents of the flask were poured out and solids were measured. 4.13 g of solution were dried, and 1.53 g of solids were obtained (37% solids). The resin solution was poured into a plastic bottle to give 134 g of solution. One glass transition temperature was observed at −31.9° C. using DSC according to the method described above with a scan from −100° C. to 150° C.

This oligomer can be incorporated into a pressure sensitive adhesive composition, for example, that is prepared from components comprising isooctyl acrylate.

### Illustrative Oligomer Example 1

### Random Copolymer of 80% by weight Methyl Methacrylate, 20% by weight Preparative Example 2

Comparative Oligomer Example 1 was made according to the method of Oligomer Example 1 with the exception that Preparative Example 2 was used instead of Preparative Example 1. After the batch was dried, it was ground to a powder. The molecular weight of the oligomer was determined by GPC (THF, EMD Omnisolve, 2c PL-Gel-2 300×7.5 mm, polystyrene standard): Mw=68220, Mn=48290, and a polydispersity of 1.41.

### Preparative Example 3

### Heptafluorobutyl Methacrylate

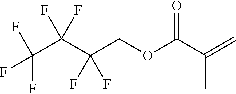 [SMILES: C=C(C)C(=O)OCC(F)(F)C(F)(F)C(F)(F)F]

Heptafluorobutanol (1890 grams, 9.45 moles), 30 grams of 95% sulfuric acid, 1.8 grams of phenothiazine, 1.5 grams of MEHQ were placed in a 3 liter flask that was fitted with an overhead stirrer, thermocouple, and an addition funnel. The reaction was heated to 55° C., and at that time the addition of methacrylic anhydride (1527 grams, 9.91 moles) was begun. The batch exothermed to 65° C., and the addition was adjusted to keep the reaction temperature at 65° C. At this time the set point of the controller was raised to 65° C. The addition of methacrylic anhydride was completed in 2.5 hours. The reaction mixture was then heated at 65° C. for 3 hours and then allowed to cool to room temperature. Analysis by gas chromatography (GC) indicated the material to be 0.4% unreacted heptafluorobutanol, 0.9% heptafluorobutyl acetate, 63.6 desired heptafluorobutyl methacrylate, 30.6% methacrylic acid, and 0.4 unreacted methacrylic anhydride.

1800 grams of water was added, and the batch was stirred for 30 minutes. The pH was measured at less than 2; analysis by GC showed the material to be 1.0% heptafluorobutyl acetate, 70.9 desired heptafluorobutyl methacrylate, 22.9% methacrylic acid, and 1.4% unreacted methacrylic anhydride. The black water phase was split off from the translucent olive/brown fluorochemical phase; 3006 grams of fluorochemical phase was obtained.

Another 1800 grams of water was added to the fluorochemical phase, and the batch was stirred for 30 minutes. The pH was measured at less than 2; analysis by GC showed the material to be 1.1% heptafluorobutyl acetate, 74.7% desired heptafluorobutyl methacrylate, 19% methacrylic acid, and 1.4% unreacted methacrylic anhydride. The light green water phase was split off from the translucent green flluorochemical phase; 2840 grams of fluorochemical phase was obtained.

The batch was allowed to split, and the translucent amethyst fluorochemical bottom phase was split off and saved. The fluorochemical phase was then stirred for 30 minutes with a mixture of 285 grams of potassium hydroxide and 1800 grams of water. The bottom raspberry colored fluorochemical phase was split off to give 2537 grams of the crude product; analysis by GC showed the material to be 1.3% heptafluorobutyl acetate, 88.3% desired heptafluorobutyl methacrylate, 6.7% methacrylic acid, and 1.4 unreacted methacrylic anhydride.

For the next wash the batch was added to 85 g of potassium carbonate dissolved in 1800 g of water and stirred for 30 min with the previously washed FC product. GC showed the material to be 1.3% heptafluorobutyl acetate and 94.4% desired heptafluorobutyl methacrylate. Methacrylic acid and unreacted methacrylic anhydride were not detected. The pH of the water layer was measured at 10-11. The product weighed 2275 grams. This material was washed again with 1800 grams of water for 30-minutes. The pH of the water layer was measured at 7-8. A total of 2235 grams of the product was isolated after separation of the water layer.

The crude heptafluorobutyl methacrylate was added to a 3 liter flask fitted with a distillation head and a thermocouple. More inhibitor (3 grams of phenothiazine and 0.7 gram of MEHQ) were added to the distillation pot. The acrylate was distilled to give 156 of precut distilling at 142 mm Hg at a head temperature of 80° C.-86° C. (88% desired methacrylate). The desired material distilled at 86° C.-° C. at 131 mm Hg; a total of 1934 grams of heptafluorobutyl methacrylate were obtained.

### Example 1, Illustrative Example 1, Comparative Example 1

Oligomer Example 1 and Illustrative Oligomer Example 1 were extruded with a PVDF HFP copolymer (obtained from 3M Company, St. Paul, Minn., under the trade designation “DYNEON 11010”) using a 20/40 mm co-rotating twin screw extruder obtained from Brabender, Duisburg, Germany, equipped with a die and cast wheel to produce films that were 6 inches wide and 0.001 inch thick between two polyester liners. The die and extruder temperatures were 480° F. (249° C.). The extruders were set up with two feed hoppers to dispense the PVDF HFP copolymer and the ultraviolet light-absorbing oligomer individually. The extrusion rates of the PVDF HFP and ultraviolet light-absorbing oligomer were 950 grams/hour and 50 grams/hour, respectively. The oligomers used for Example 1 and Illustrative Example 1 are shown in Table 1, below. The final UVA wt % in the film referred to in Table 1 refers to the wt % of the active UV absorbing unit in the oligomer. Oligomers were added at 5% by weight to provide 1% by weight of the active UV absorbing monomeric unit in the film. In Comparative Example 1, 1% by weight of UV absorber 2-[4-[(2-hydroxy-3-(2′-ethyl) hexyl)oxy]-2-hydroxyphenyl]-4,6-bis(2,4-dimethylphenyl)-1,3,5-triazine (obtained from BASF, Florham Park, N.J., under the trade designation “TINUVIN 405”) was also extrusion compounded into PVDF at similar process conditions as described above. The PVDF was obtained from 3M Company under the trade designation “DYNEON 6008”

TABLE 1

| | | Wt. % | Wt % | |
|---|---|---|---|---|
| Film | | active | MMA from | Wt. % |
| Example | UVA | UVA | oligomer | PVDF/HFP |
| Example 1 | Olig. Ex. 1 | 1 | 4 | 95 |
| Ill. Ex. 1 | Ill. Olig. Ex. 1 | 1 | 4 | 95 |
| Comp. Ex. 1 | “TINUVIN 405” | 1 | Not applicable | 99 |

Average transmission for Example 1, Illustrative Example 1, and Comparative Example 1 were measured using a “LAMBDA 950” Spectrophotometer obtained from Lambda Scientific before and after Accelerated Ultraviolet Light Exposure for one interval according to the method described above. The results are shown in Table 2, below.

TABLE 2

| | Avg. Transmission | | Avg. Transmission | | Avg. Transmission | |
|---|---|---|---|---|---|---|
| | 300 nm-380 nm (%) | | 380 nm-450 nm (%) | | 400 nm to 750 nm (%) | |
| Film | | 1 | | 1 | | 1 |
| Example | initial | interval | initial | interval | initial | interval |
| Example 1 | 6.7 | 7.3 | 80.1 | 81.2 | 90.2 | 90.4 |
| Illustrative Ex. 1 | 15.1 | 29.3 | 88.3 | 87.6 | 92.7 | 92.6 |
| Comp. Ex. 1 | 34.8 | 89.7 | 92.3 | 92.7 | 93.5 | 93.4 |

Average absorbance at 360 nm for Example 1, Illustrative Example 1, and Comparative Example 1 were measured using a “LAMBDA 950” Spectrophotometer obtained from Lambda Scientific before and after Accelerated Ultraviolet Light Exposure for one interval according to the method described above. The results are shown in Table 3, below.

TABLE 3
Absorbance at 360 nm

| Film | | | % UVA |
|---|---|---|---|
| Example | initial | 1 interval | retention |
| Comp. Ex. 1 | 0.288 | 0.045 | 15% |
| Illustrative Ex. 1 | 0.701 | 0.375 | 53% |
| Example 1 | 1.349 | 1.289 | 96% |

Various modifications and alterations of this disclosure may be made by those skilled the art without departing from the scope and spirit of the disclosure, and it should be understood that this invention is not to be unduly limited to the illustrative embodiments set forth herein.

1. A composition comprising a blend of a fluoropolymer and an ultraviolet light-absorbing oligomer, wherein the ultraviolet light-absorbing oligomer comprises:
a first divalent unit represented by formula: 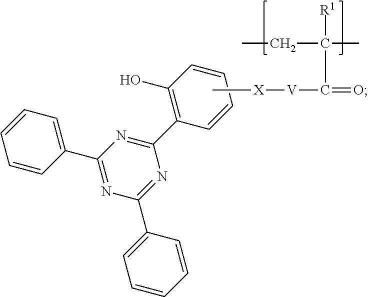
and
a second divalent unit represented by formula: 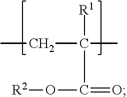
wherein
each R¹ is independently hydrogen or methyl;
V is O or NH;
X is a bond, alkylene, or alkyleneoxy, wherein the alkylene or alkyleneoxy have from 1 to 10 carbon atoms and are optionally interrupted by one or more —O— groups and optionally substituted by a hydroxyl group; and
R² is alkyl having from 1 to 4 carbon atoms.

2. The composition of claim 1, wherein the composition is an extruded film.

3. The composition of claim 1, further comprising a hindered amine light stabilizer.

4. The composition of claim 1, wherein the ultraviolet light-absorbing oligomer further comprises a third divalent unit represented by formula: 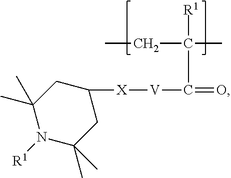
wherein
R¹ is independently hydrogen or methyl;
X is a bond, alkylene, or alkyleneoxy, wherein the alkylene or alkyleneoxy have from 1 to 10 carbon atoms and are optionally interrupted by one or more —O— groups and optionally substituted by a hydroxyl group;
V is O or NH; and
R³ is hydrogen, alkyl, oxy, alkoxy, or alkanone.

5. The composition of claim 1, wherein the ultraviolet light-absorbing oligomer further comprises a fifth divalent unit represented by formula: 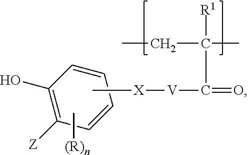
wherein
R¹ is independently hydrogen or methyl;
V is O or NH;
X is a bond, alkylene, or alkyleneoxy, wherein the alkylene or alkyleneoxy have from 1 to 10 carbon atoms and are optionally interrupted by one or more —O— groups and optionally substituted by a hydroxyl group;
R is alkyl having from one to four carbon atoms;
n is 0 or 1; and
Z is a benzoyl group optionally substituted by hydroxyl, alkyl, halogen, or hydroxyl or a 2H-benzotriazol-2-yl group optionally substituted by one or more halogens.

6. The composition of claim 1, wherein the ultraviolet light-absorbing oligomer further comprises a fourth divalent unit represented by formula: 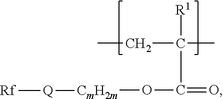
wherein
Rf represents a fluoroalkyl group having from 1 to 8 carbon atoms optionally interrupted by one —O— group, or Rf represents a polyfluoropolyether group;
R¹ is independently hydrogen or methyl;
Q is a bond, —SO₂—N(R⁵)—, or —C(O)—N(R⁵)—, wherein R is alkyl having from 1 to 4 carbon atoms or hydrogen; and
m is an integer from 0 to 10.

7. The composition of claim 1, wherein the ultraviolet light-absorbing oligomer is in the composition in an amount ranging from 1 percent to 25 percent by weight, based on the total weight of the composition.

8. The composition of claim 1, wherein in the second divalent unit, R¹ and R² are both methyl.

9. The composition of claim 1, further comprising a second, different oligomer comprising the second divalent units and at least one of:
a third divalent unit comprising a pendent 2,2,6,6-tetramethylpiperidinyl group, wherein the nitrogen of the pendent 2,2,6,6-tetramethylpiperidinyl group is substituted by hydrogen, alkyl, alkoxy, or alkanone; or
a fifth divalent unit comprising a pendent ultraviolet absorbing group selected from a benzophenone and a benzotriazole.

10. The composition of claim 1, wherein the fluoropolymer is present in the blend in an amount of at least 70 percent by weight, based on the total weight of the blend.

11. The composition of claim 1, wherein the blend further comprises poly(methyl methacrylate).

12. The composition of claim 1, wherein the fluoropolymer is selected from the group consisting of ethylene-tetrafluoroethylene copolymer, a tetrafluoroethylene-hexafluoropropylene copolymer, a tetrafluoroethylene-hexafluoropropylene-vinylidene fluoride copolymer, or polyvinylidene fluoride.

13. An article comprising the composition of claim 1, wherein the article is a photovoltaic device, vehicle wrap, graphic film, architectural film, or window film.

14. An ultraviolet light-absorbing oligomer comprising:
a first divalent unit represented by formula: 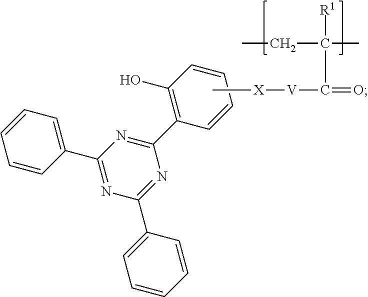
and
a second divalent unit represented by formula: 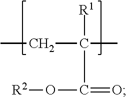
wherein
each R¹ is independently hydrogen or methyl;
V is O or NH;
X is a bond, alkylene, or alkyleneoxy, wherein the alkylene or alkyleneoxy have from 1 to 10 carbon atoms and are optionally interrupted by one or more —O— groups and optionally substituted by a hydroxyl group; and
R² is alkyl having from 1 to 22 carbon atoms.

15. A pressure sensitive adhesive comprising the ultraviolet light-absorbing oligomer of claim 14.

16. The ultraviolet light-absorbing oligomer of claim 14, wherein the ultraviolet light-absorbing oligomer further comprises a third divalent unit represented by formula: 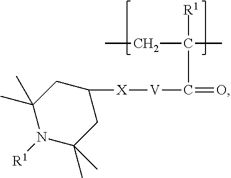
wherein
R¹ is independently hydrogen or methyl;
X is a bond, alkylene, or alkyleneoxy, wherein the alkylene or alkyleneoxy have from 1 to 10 carbon atoms and are optionally interrupted by one or more —O— groups and optionally substituted by a hydroxyl group;
V is O or NH; and
R³ is hydrogen, alkyl, oxy, alkoxy, or alkanone.

17. The ultraviolet light-absorbing oligomer of claim 14, wherein the ultraviolet light-absorbing oligomer further comprises a fifth divalent unit represented by formula: 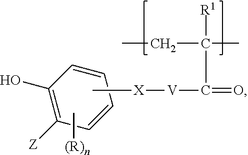
wherein
R¹ is independently hydrogen or methyl;
V is O or NH;
X is a bond, alkylene, or alkyleneoxy, wherein the alkylene or alkyleneoxy have from 1 to 10 carbon atoms and are optionally interrupted by one or more —O— groups and optionally substituted by a hydroxyl group;
R is alkyl having from one to four carbon atoms;
n is 0 or 1; and
Z is a benzoyl group optionally substituted by hydroxyl, alkyl, halogen, or hydroxyl or a 2H-benzotriazol-2-yl group optionally substituted by one or more halogens.

18. The ultraviolet light-absorbing oligomer of claim 14, wherein the ultraviolet light-absorbing oligomer further comprises a fourth divalent unit represented by formula: 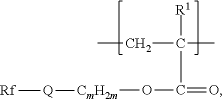
wherein
Rf represents a fluoroalkyl group having from 1 to 8 carbon atoms optionally interrupted by one —O— group, or Rf represents a polyfluoropolyether group;
R¹ is independently hydrogen or methyl;
Q is a bond, —SO₂—N(R⁵)—, or —C(O)—N(R⁵)—, wherein R is alkyl having from 1 to 4 carbon atoms or hydrogen; and
m is an integer from 0 to 10.

19. The ultraviolet light-absorbing oligomer of claim 14, wherein the ultraviolet light-absorbing oligomer further comprises a sixth divalent unit comprising a pendent carboxylic acid, hydroxyl, aminocarbonyl, alkylaminocarbonyl, or dialkylaminocarbonyl group, wherein the alkyl in the alkylaminocarbonyl or dialkylaminocarbonyl is optionally substituted by hydroxyl.

20. The composition of claim 1, wherein the ultraviolet light-absorbing oligomer has a number average molecular weight of less than 20,000 grams per mole and wherein R¹ and R² are both methyl.
